(19) United States
(12) Patent Application Publication
(10) Pub. No.: US 2016/0058895 A1
(43) Pub. Date: Mar. 3, 2016

(54) RADIOLABELED GNRH ANTAGONISTS AS PET IMAGING AGENTS

(71) Applicant: OSLO UNIVERSITETSSYKEHUS HF, Olso (NO)

(72) Inventors: Dag Erlend Olberg, Oslo (NO); Ira Hebold Haraldsen, Oslo (NO); Jo Klaveness, Oslo (NO)

(73) Assignee: Oslo Universitetssykehus HF, Oslo (NO)

(21) Appl. No.: 14/784,998

(22) PCT Filed: Apr. 18, 2014
(86) PCT No.: PCT/IB2014/001726

§ 371 Date: Oct. 16, 2015

Related U.S. Application Data
(60) Provisional application No. 61/813,756, filed on Apr. 19, 2013.

(51) Int. Cl. A61K 51/04; C07D 307/68; C07D 405/12

(52) U.S. Cl. CPC A61K 51/0459; C07D 405/12; C07D 307/68; A61K 51/0419; C07B 59/002

(57) ABSTRACT

Provided herein is technology relating to imaging agents for positron emission tomography (PET) and particularly, but not exclusively, to a gonadotropin-releasing hormone (GnRH) antagonist radiolabeled with positron emitting nuclides and to methods of visualizing GnRH receptors in the central nervous system by PET from administration of such compounds to warm-blooded animals for diagnostic purposes.

This application claims priority to U.S. provisional patent application Ser. No. 61/813,756, filed Apr. 19, 2013, which is incorporated herein by reference in its entirety.

### FIELD OF INVENTION

Provided herein is technology relating to imaging agents for positron emission tomography (PET) and particularly, but not exclusively, to a gonadotropin-releasing hormone (GnRH) antagonist radiolabeled with positron emitting nuclides and to methods of visualizing GnRH receptors in the central nervous system by PET from administration of such compounds to warm-blooded animals for diagnostic purposes.

### BACKGROUND

Gonadotropin-releasing hormone (GnRH), also known as luteinizing hormone-releasing hormone is a decapeptide (pGlu¹-His²-Trp³-Ser⁴-Tyr⁵-Gly⁶-Leu⁷-Arg⁸-Pro⁹-Gly¹⁰-NH₂) that plays an important role in human reproduction. GnRH is released form the hypothalamus and acts on the pituitary gland to stimulate the biosynthesis and release of luteinizing hormone (LH) and follicle-stimulating hormone (FSH). LH released from the pituitary gland is responsible for the regulation of gonadal steroid production in both males and females, while FSH regulates spermatogenesis in males and follicular development in females. However, there are occasional reports that GnRH has unexpected effects or is present in nonreproductive tissues, forcing us to reconsider the role of GnRH in the physiology of living beings.

The hippocampus is one of the first brain substructures to be affected in Alzheimer's disease (AD) and expresses high levels of GnRH receptors. In the human hippocampus, pyramidal neurons express GnRH receptors. Similarly, GnRH receptor-immunoreactive neurons were found almost exclusively within the pyramidal cell layer, dentate gyrus, and indusium griseum of the mouse and sheep. Because GnRH is likely to be elevated post-menopause due to the loss of estrogen negative feedback, the effect of GnRH on these neurons may constitute a component of the neurodegenerative pathology that accompanies Alzheimer's disease. It is notable that hippocampal spinophilin, a reliable dendritic spine marker, is significantly decreased in response to high doses of GnRH. Several peer-reviewed papers report a significant correlation between cognitive decline and dysfunction in the CNS GnRH system. Accordingly, diagnostic tools for evaluating GnRH receptor activity in the CNS can provide useful and prognostic valuable information with respect to an individual risk for developing AD.

Conventional diagnosis of AD primarily relies on patient history, clinical observation, and cognitive tests, which only become reliable in the later stages of AD. Conventional diagnostic agents such as ¹¹C-Pittsburgh compound B (PiB) and its ¹⁸F-fluorinated analogs provide in some cases for non-invasive imaging of beta-amyloid plaques in neuronal tissue using PET; however, definitive and early AD diagnosis with these agents is not always achieved. Peptides do not readily cross the blood-brain barrier (BBB) and a radiolabelled GnRH peptide would not simply cross the BBB. In addition, several laboratories have reported orally active nonpeptide GnRH receptor antagonists intended for therapeutic purposes; however, no information is provided with respect to their brain uptake.

### SUMMARY

Some small organic molecules have characteristics similar to those described by Lipinski as being predictive of solubility and permeability (Lipinski et al. (1997) “Experimental and computational approaches to estimate solubility and permeability in drug discovery and development settings” Adv Drug Deilv Rev 23: 3-25). In particular, orally active furamide-based GnRH antagonists are compounds that satisfy the general requirements of a CNS active drug. Accordingly, the present technology describes furamide based GnRH antagonists labeled with PET radioisotopes such as fluorine-18 (t_{1/2}=109.7 minutes).

PET images of rat brain show that embodiments of the imaging agents provided herein cross the blood-brain barrier. These GnRH analogs (e.g., peptidomimetics) demonstrate significant uptake in the central parts of the rat brain. In particular, PET images (e.g., 90-minute summed PET images) of the brain of a Sprague-Dawley rat injected with an [¹⁸F] compound according to an embodiment of the technology show accumulation of the compound in the central regions of the brain. Additional experiments showed that plaques in an AD mouse cortex after treatment with a GnRH blocker had a looser structure than the same plaques before treatment, indicating that treatment induced solubility of amyloid Aβ-40.

Based on these observations of blood-brain barrier permeability, additional compounds were synthesized to enhance GnRH receptor traceability in the hippocampus and other important non-reproductive brain areas. This class of novel PET radiotracers provides new diagnostic agents for in vivo imaging of the GnRH dysfunction associated with pre-symptomatic AD.

For example, in some embodiments, the technology is related to a compound having a structure according to one of the following:

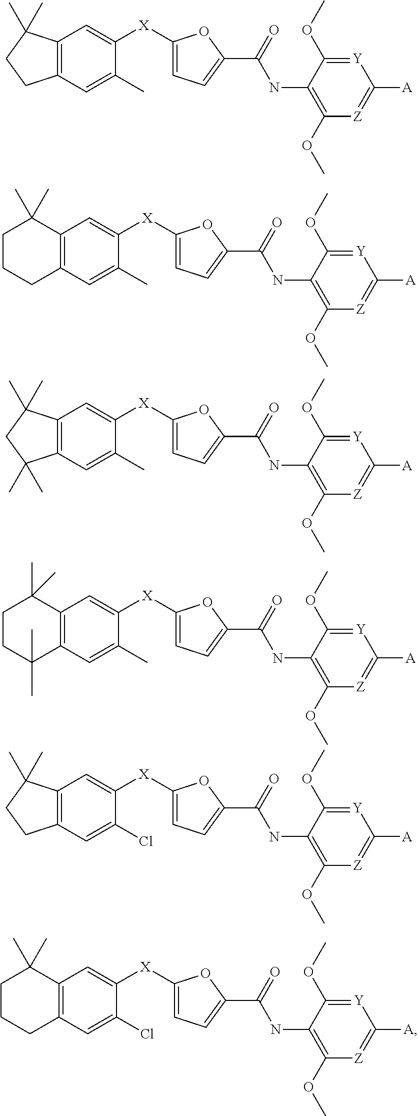

or a salt, a free base, or a combination thereof, wherein X is O or C; Y is C or N; Z is C or N; and A is one of the following:

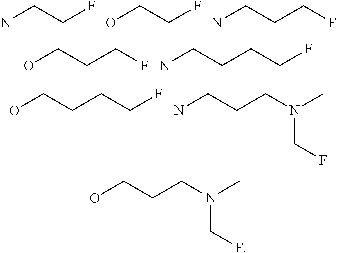

In some embodiments, the compound has a structure according to one of the following:

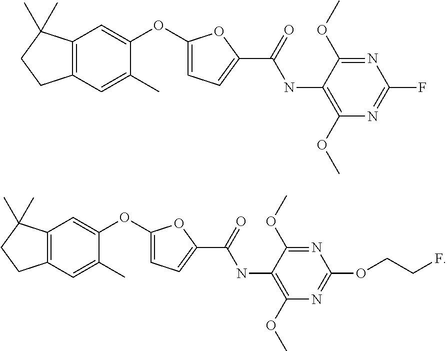 [SMILES: COc1nc(F)nc(OC)c1NC(=O)c1ccc(Oc2cc3c(cc2C)CCC3(C)C)o1 ; COc1nc(OCCF)nc(OC)c1NC(=O)c1ccc(Oc2cc3c(cc2C)CCC3(C)C)o1]

In some embodiments, the compound comprises a ¹⁹F or ¹⁸F, e.g., at the position designated by F in the structures above.

In addition, the technology is related to embodiments of methods for imaging a subject, the method comprising administering a compound described herein and imaging the patient using positron emission tomography. In some embodiments, the subject has or is suspected of having Alzheimer's disease, a plaque-associated disease, or a condition associated with the activity of gonadotropin-releasing hormone or a gonadotropin-releasing hormone receptor.

In some embodiments are provided methods of imaging a tissue. In some embodiments, tissue imaging methods comprise contacting a tissue to be imaged with a compound as described herein and imaging the tissue. In some embodiments, the tissue is nervous tissue; in some embodiments, the tissue is central nervous system tissue; in some embodiments, the tissue is brain tissue; in some embodiments, the tissue comprises a gonadotropin-releasing hormone receptor. In some embodiments, the tissue has, or is suspected of having, a disease-associated plaque.

In some embodiments, the technology is related to a composition comprising a compound as described herein and a pharmaceutically acceptable carrier suitable for administration to a subject. Moreover, the technology provided embodiments of uses such as use of a composition comprising a compound as described herein as an imaging agent. Additional embodiments provide use of a composition comprising a compound as described herein as an imaging agent for the diagnosis of Alzheimer's disease.

For instance, in some embodiments the technology provides a compound having a structure that is

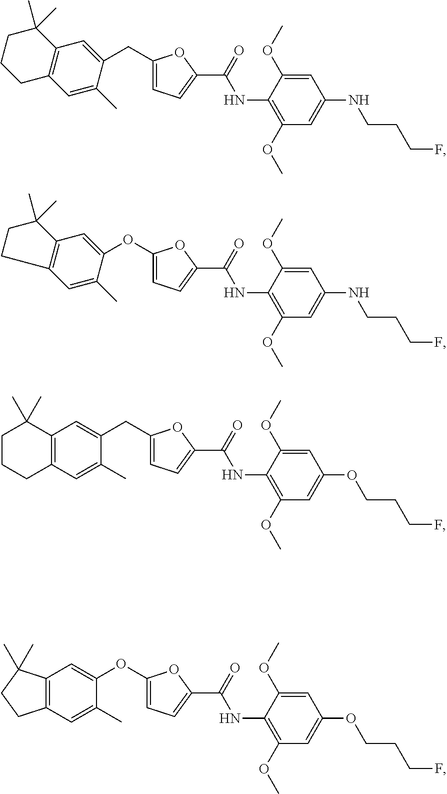

or a salt, a free base, or a combination thereof. In some embodiments, the F is ¹⁹F or ¹⁸F. The compounds have defining characteristics such as partition coefficients, receptor affinities, etc. For instance, in some embodiments the compound has a Log P value in a phosphate-buffered saline (pH 7.4) and n-octanol system that is from 1.2 to 2.0 (e.g., 1.3, 1.4, 1.5, 1.6, 1.7, 1.8, 1.9). In some embodiments, a compound is provided having a receptor affinity (10 for the human GnRH receptor of 0.1 to 6.0 nM (e.g., 0.2, 0.5, 1.0, 1.5, 2.0, 2.5, 3.0, 3.5, 4.0, 4.5, 5.0, 5.5). Related embodiments provide use of a composition comprising a compound as provided herein as an imaging agent (e.g., for the diagnosis of Alzheimer's disease). In some embodiments, the group designated by the “A” in the structures above does not comprise a fluorine group. For example, in some embodiment the compounds given by the structure above comprise a group at the “A” position that is H, alkyl, aryl, alkylaryl, amino, or alkoxy.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a time activity curve showing the uptake of an embodiment of a radioactive compound according to the technology provided herein. The upper curve shows the time activity curve for uptake in intact brain and the lower curve shows the time activity curve for uptake in metabolite brain.

FIG. 2 is a plot showing a radiochromatogram of an embodiment of a ¹⁸F compound provided herein (e.g., [¹⁸F]SB-004-RS (upper trace)) spiked with an embodiment of the related F compound as provided herein (e.g., SB-004-RS (lower trace)).

FIG. 3 is a plot showing representative binding curves for embodiments of compounds provided herein (e.g., SB-001-RS, SB-002-RS, SB-003-RS and SB-004-RS) for the human GnRH receptor in competition with [¹²⁵I]Triptorelin. The data for SB-001-RS are shown in circles, SB-002-RS are shown in squares, SB-003-RS are shown in triangles with a vertex pointing up, and SB-004-RS are shown in triangles with a vertex pointing down.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to imaging agents for positron emission tomography (PET) and particularly, but not exclusively, to a gonadotropin-releasing hormone (GnRH) antagonist radiolabeled with positron emitting nuclides and to methods of visualizing GnRH receptors in the central nervous system by PET from administration of such compounds to warm-blooded animals for diagnostic purposes.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein the term, “in vitro” refers to an artificial environment and to processes or reactions that occur within an artificial environment. In vitro environments may include, but are not limited to, test tubes and cell cultures. The term “in vivo” refers to the natural environment (e.g., an animal or a cell) and to processes or reactions that occur within a natural environment.

As used herein, the terms “subject” and “patient” refer to any animal, such as a mammal like a dog, cat, bird, livestock, and preferably a human (e.g., a human with a disease such as obesity, diabetes, or insulin resistance).

As used herein, the term “effective amount” refers to the amount of a composition sufficient to effect beneficial or desired results. An effective amount can be administered in one or more administrations, applications, or dosages and is not intended to be limited to a particular formulation or administration route.

As used herein, the term “administration” refers to the act of giving a drug, prodrug, or other agent, or therapeutic treatment to a subject. Exemplary routes of administration to the human body can be through the eyes (ophthalmic), mouth (oral), skin (transdermal, topical), nose (nasal), lungs (inhalant), oral mucosa (buccal), ear, by injection (e.g., intravenously, subcutaneously, intratumorally, intraperitoneally, etc.), and the like.

As used herein, the term “co-administration” refers to the administration of at least two agents or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent.

As used herein, the term “pharmaceutical composition” refers to the combination of an active agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present technology.

As used herein, the terms “alkyl” and the prefix “alk-” are inclusive of both straight chain and branched chain saturated or unsaturated groups, and of cyclic groups, e.g., cycloalkyl and cycloalkenyl groups. Unless otherwise specified, acyclic alkyl groups are from 1 to 6 carbons. Cyclic groups can be monocyclic or polycyclic and preferably have from 3 to 8 ring carbon atoms. Exemplary cyclic groups include cyclopropyl, cyclopentyl, cyclohexyl, and adamantyl groups. Alkyl groups may be substituted with one or more substituents or unsubstituted. Exemplary substituents include alkoxy, aryloxy, sulfhydryl, alkylthio, arylthio, halogen, alkylsilyl, hydroxyl, fluoroalkyl, perfluoralkyl, amino, aminoalkyl, disubstituted amino, quaternary amino, hydroxyalkyl, carboxyalkyl, and carboxyl groups. When the prefix “alk” is used, the number of carbons contained in the alkyl chain is given by the range that directly precedes this term, with the number of carbons contained in the remainder of the group that includes this prefix defined elsewhere herein. For example, the term “C₁-C₄ alkaryl” exemplifies an aryl group of from 6 to 18 carbons (e.g., see below) attached to an alkyl group of from 1 to 4 carbons.

As used herein, the term “aryl” refers to a carbocyclic aromatic ring or ring system. Unless otherwise specified, aryl groups are from 6 to 18 carbons. Examples of aryl groups include phenyl, naphthyl, biphenyl, fluorenyl, and indenyl groups.

As used herein, the term “heteroaryl” refers to an aromatic ring or ring system that contains at least one ring heteroatom (e.g., O, S, Se, N, or P). Unless otherwise specified, heteroaryl groups are from 1 to 9 carbons. Heteroaryl groups include furanyl, thienyl, pyrrolyl, imidazolyl, pyrazolyl, oxazolyl, isoxazolyl, thiazolyl, isothiazolyl, triazolyl, tetrazolyl, oxadiazolyl, oxatriazolyl, pyridyl, pyridazyl, pyrimidyl, pyrazyl, triazyl, benzofuranyl, isobenzofuranyl, benzothienyl, indole, indazolyl, indolizinyl, benzisoxazolyl, quinolinyl, isoquinolinyl, cinnolinyl, quinazolinyl, naphtyridinyl, phthalazinyl, phenanthrolinyl, purinyl, and carbazolyl groups.

As used herein, the term “heterocycle” refers to a non-aromatic ring or ring system that contains at least one ring heteroatom (e.g., O, S, Se, N, or P). Unless otherwise specified, heterocyclic groups are from 2 to 9 carbons. Heterocyclic groups include, for example, dihydropyrrolyl, tetrahydropyrrolyl, piperazinyl, pyranyl, dihydropyranyl, tetrahydropyranyl, dihydrofuranyl, tetrahydrofuranyl, dihydrothiophene, tetrahydrothiophene, and morpholinyl groups.

Aryl, heteroaryl, or heterocyclic groups may be unsubstituted or substituted by one or more substituents selected from the group consisting of C₁₋₆ alkyl, hydroxy, halo, nitro, C₁₋₆ alkoxy, C₁₋₆ alkylthio, trifluoromethyl, C₁₋₆ acyl, arylcarbonyl, heteroarylcarbonyl, nitrile, C₁₋₆ alkoxycarbonyl, alkaryl (where the alkyl group has from 1 to 4 carbon atoms), and alkheteroaryl (where the alkyl group has from 1 to 4 carbon atoms).

As used herein, the term “alkoxy” refers to a chemical substituent of the formula —OR, where R is an alkyl group. By “aryloxy” is meant a chemical substituent of the formula —OR′, where R′ is an aryl group.

As used herein, the term “C_{x-y} alkaryl” refers to a chemical substituent of formula —RR′, where R is an alkyl group of x to y carbons and R′ is an aryl group as defined elsewhere herein.

As used herein, the term “C_{x-y} alkheteraryl” refers to a chemical substituent of formula RR″, where R is an alkyl group of x to y carbons and R″ is a heteroaryl group as defined elsewhere herein.

As used herein, the term “halide” or “halogen” or “halo” refers to bromine, chlorine, iodine, or fluorine.

As used herein, the term “non-vicinal O, S, or N” refers to an oxygen, sulfur, or nitrogen heteroatom substituent in a linkage, where the heteroatom substituent does not form a bond to a saturated carbon that is bonded to another heteroatom.

For structural representations where the chirality of a carbon has been left unspecified it is to be presumed by one skilled in the art that either chiral form of that stereocenter is possible.

### Embodiments of the Technology

### Imaging Agents

Provided herein are novel ¹⁸F-labelled small molecule GnRH antagonists that have potent activity and favorable pharmacological properties for in vivo visualization of GnRH receptors in CNS. The compounds are based on the furamide pharmacophore. The compounds are designed to reduce polar surface area (PSA) and increase log P to improve CNS uptake and potency (Kᵢ).

In some embodiments, the imaging agent has the structure according to:

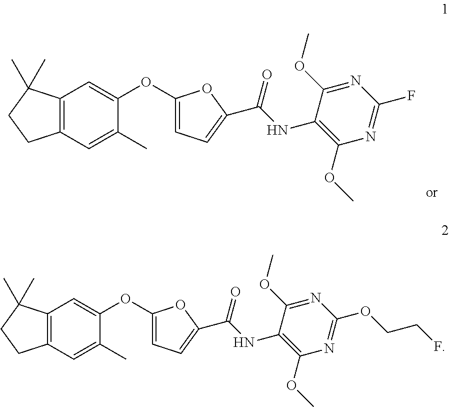 [SMILES: COc1nc(F)nc(OC)c1NC(=O)c1ccc(Oc2cc3c(cc2C)CCC3(C)C)o1 ; COc1nc(OCCF)nc(OC)c1NC(=O)c1ccc(Oc2cc3c(cc2C)CCC3(C)C)o1]

Test data demonstrated that Compound 1 showed CNS uptake (≈0.4% ID/g at 10 minutes) and that Compound 2 is potent and has good in vivo stability.

In some embodiments, compounds are preferably synthesized according to a scheme such as follows:

### 1) Synthesis of Precursor for Compound 1

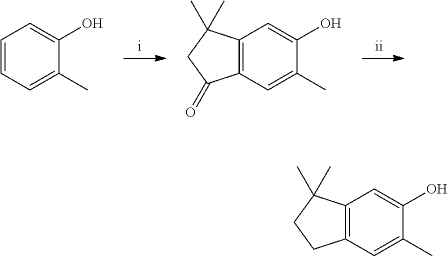

Reagents and conditions: (i) 3,3-dimethylacrylic acid, polyphosphoric acid, 105° C.; (ii) H₂, Pd/C, H₂SO₄, MeOH.

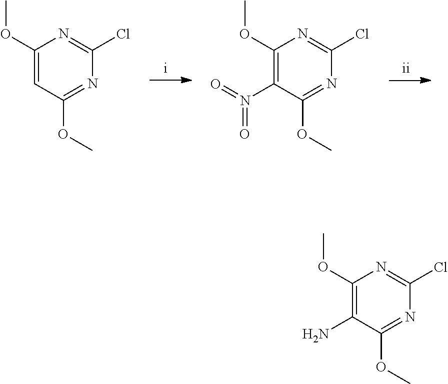

Reagents and conditions: (i) tetramethylammonium nitrate, triflic anhydride, DCM; Fe(s), EtOH, NH₄Cl (sat)

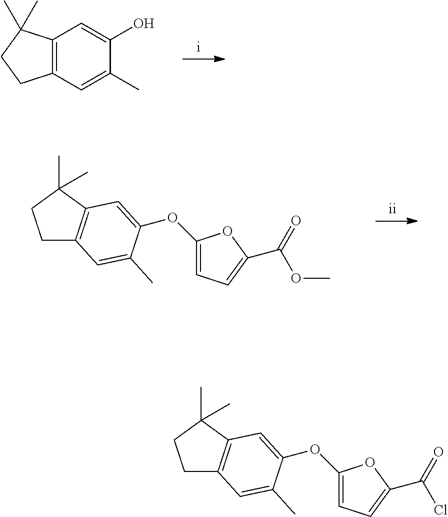 [SMILES: Cc1cc2c(cc1O)C(C)(C)CC2 ; COC(=O)c1ccc(Oc2cc3c(cc2C)CCC3(C)C)o1 ; Cc1cc2c(cc1Oc1ccc(C(=O)Cl)o1)C(C)(C)CC2]

Reagents and conditions: (i) methyl 5-bromo-2-furoate, Cs₂CO₃, DMF; (ii) a) NaOH, MeOH; b) SOCl₂, reflux

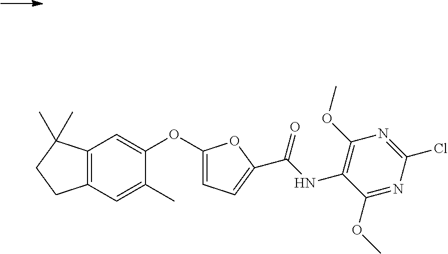 [SMILES: COc1nc(Cl)nc(OC)c1NC(=O)c1ccc(Oc2cc3c(cc2C)CCC3(C)C)o1]

Reagents and conditions: DCM, Pyridine

### 2) Radiolabeling of Precursor for Compound 1

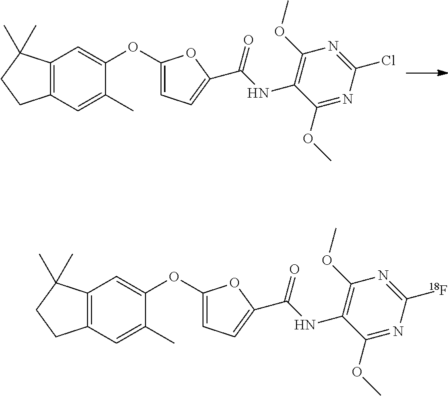 [SMILES: COc1nc(Cl)nc(OC)c1NC(=O)c1ccc(Oc2cc3c(cc2C)CCC3(C)C)o1 ; COc1nc([18F])nc(OC)c1NC(=O)c1ccc(Oc2cc3c(cc2C)CCC3(C)C)o1]

Reagents and conditions: DMSO, [¹⁸F]KF/K222, DMSO, 120° C. 10-15% radiochemical yield (non-optimized—RCP>95%)

Similar syntheses provide the following 5-indanol/benzene based compounds that are embodiments of the technology provided herein.

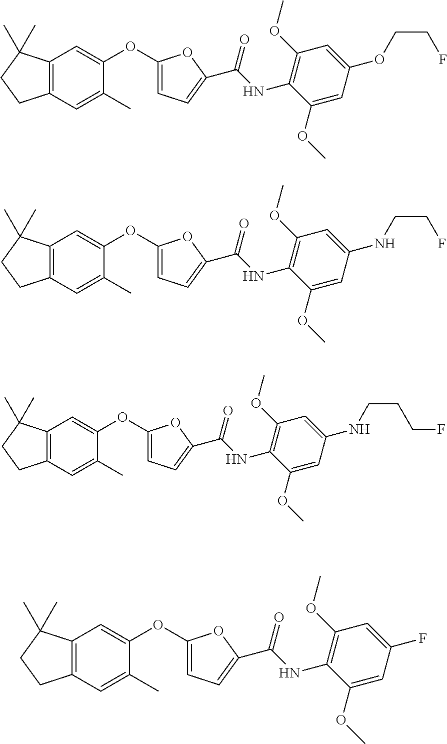 [SMILES: COc1cc(OCCF)cc(OC)c1NC(=O)c1ccc(Oc2cc3c(cc2C)CCC3(C)C)o1 ; COc1cc(NCCF)cc(OC)c1NC(=O)c1ccc(Oc2cc3c(cc2C)CCC3(C)C)o1 ; COc1cc(NCCCF)cc(OC)c1NC(=O)c1ccc(Oc2cc3c(cc2C)CCC3(C)C)o1 ; COc1cc(F)cc(OC)c1NC(=O)c1ccc(Oc2cc3c(cc2C)CCC3(C)C)o1]

These compounds reduce PSA with about 20 compared to compounds 1 and 2.
Similar syntheses provide the following tetralin-/benzene based compounds that are embodiments of the technology provided herein.

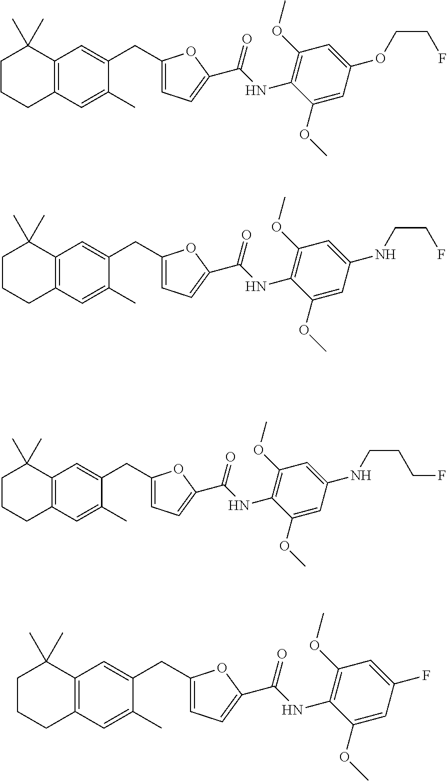 [SMILES: COc1cc(OCCF)cc(OC)c1NC(=O)c1ccc(Cc2cc3c(cc2C)CCCC3(C)C)o1 ; COc1cc(NCCF)cc(OC)c1NC(=O)c1ccc(Cc2cc3c(cc2C)CCCC3(C)C)o1 ; COc1cc(NCCCF)cc(OC)c1NC(=O)c1ccc(Cc2cc3c(cc2C)CCCC3(C)C)o1 ; COc1cc(F)cc(OC)c1NC(=O)c1ccc(Cc2cc3c(cc2C)CCCC3(C)C)o1]

These compounds reduce PSA with about 30 to 40 compared to compounds 1 and 2.
Similar syntheses provide the following tetralin/pyridine based compounds that are embodiments of the technology provided herein.

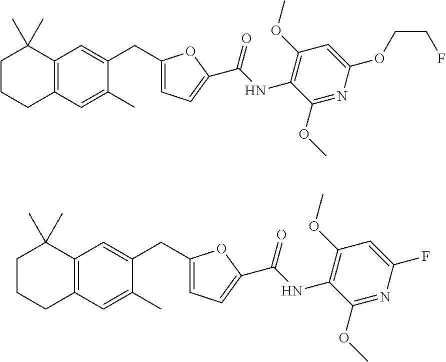

These compounds reduce PSA with about 10 to 15 compared to compounds 1 and 2.
In some embodiments, the compounds have a structure according to one of the following:

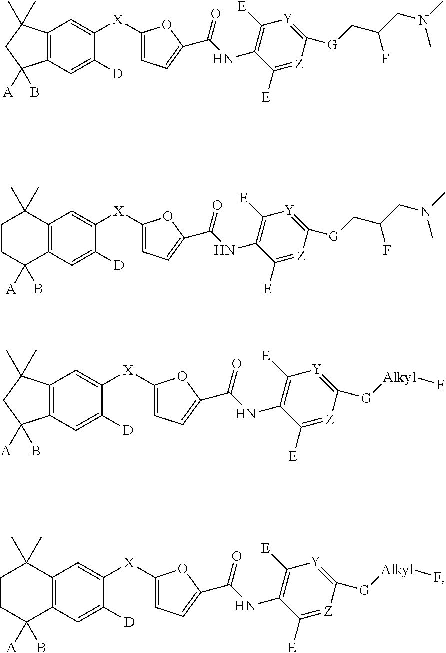

in which X is C or O; Y is C or N; Z is C or N; G is N or O; A and B are H or methyl; D is methyl or Cl; E is methoxy; and Alkyl may be 2 to 4 carbons. Furthermore, in some embodiments, F is fluorine-18 or Fluorine-19.

### Uses of Imaging Agents

The imaging agents of the present technology find many uses. In particular, the imaging agents of the present technology find use as imaging agents within nuclear medicine imaging protocols (e.g., PET imaging, SPECT imaging).

In preferred embodiments, the imaging agents of the present technology are useful as imaging agents within PET imaging studies. PET is the study and visualization of human physiology by electronic detection of short-lived positron emitting radiopharmaceuticals. It is a non-invasive technology that quantitatively measures metabolic, biochemical, and functional activity in living tissue.

The PET scan is a vital method of measuring body function and guiding disease treatment. It assesses changes in the function, circulation, and metabolism of body organs. Unlike MRI (Magnetic Resonance Imaging) or CT (Computed Tomography) scans that primarily provide images of organ anatomy, PET measures chemical changes that occur before visible signs of disease are present on CT and MRI images.

PET visualizes behaviors of trace substances within a subject (e.g., a living body) having a radioimaging agent administered therein by detecting a pair of photons occurring as an electron/positron annihilation pair and moving in directions opposite from each other (see, e.g., U.S. Pat. No. 6,674,083, herein incorporated by reference in its entirety). A PET apparatus is equipped with a detecting unit having a number of small-size photon detectors arranged about a measurement space in which the subject is placed. The detecting unit detects frequencies of the generation of photon pairs in the measurement space on the basis of the stored number of coincidence-counting information items, or projection data, and then stores photon pairs occurring as electron/positron annihilation pairs by coincidence counting and reconstructs an image indicative of spatial distributions. The PET apparatus plays an important role in the field of nuclear medicine and the like, whereby biological functions and higher-order functions of brains can be studied by using it. Such PET apparatuses can be roughly classified into two-dimensional PET apparatuses, three-dimensional PET apparatuses, and slice-septa-retractable type three-dimensional PET apparatuses.

In general, a PET detector or camera typically consists of a polygonal or circular ring of radiation detection sensors placed around a patient area (see, e.g., U.S. Pat. No. 6,822,240, herein incorporated by reference in its entirety). Radiation detection begins by injecting isotopes with short half-lives into a patient's body placed within the patient area. The isotopes are absorbed by target areas within the body and emit positrons. In the human body, the positrons annihilate with electrons. As a result thereof, two essentially monoenergetic gamma rays are emitted simultaneously in opposite directions. In most cases the emitted gamma rays leave the body and strike the ring of radiation detectors.

The ring of detectors includes typically an inner ring of scintillation crystals and an outer ring of light detectors, e.g., photomultiplier tubes. The scintillation crystals respond to the incidence of gamma rays by emitting a flash of light (photon energy), so-called scintillation light, which is then converted into electronic signals by a corresponding adjacent photomultiplier tube. A computer, or similar, records the location of each light flash and then plots the source of radiation within the patient's body by comparing flashes and looking for pairs of flashes that arise simultaneously and from the same positron-electron annihilation point. The recorded data is subsequently translated into a PET image. A PET monitor displays the concentration of isotopes in various colors indicating level of activity. The resulting PET image then indicates a view of neoplasms or tumors existing in the patient's body.

Such detector arrangement is known to have a good energy resolution, but relatively bad spatial and temporal resolutions. Early PET detectors required a single photomultiplier tube to be coupled to each single scintillation crystal, while today, PET detectors allow a single photodetector to serve several crystals, see e.g. U.S. Pat. Nos. 4,864,138; 5,451,789; and 5,453,623, each herein incorporated by reference in their entireties). In such manner the spatial resolution is improved or the number of photodetectors needed may be reduced.

Single Photon Emission Computed Tomography (SPECT) is a tomographic nuclear imaging technique producing cross-sectional images from gamma ray emitting radiopharmaceuticals (single photon emitters or positron emitters). SPECT data are acquired according to the original concept used in tomographic imaging: multiple views of the body part to be imaged are acquired by rotating the Anger camera detector head(s) around a craniocaudal axis. Using backprojection, cross-sectional images are then computed with the axial field of view (FOV) determined by the axial field of view of the gamma camera. SPECT cameras are either standard gamma cameras that can rotate around the patient's axis or consist of two or even three camera heads to shorten acquisition time. Data acquisition is over at least half a circle)(180° (used by some for heart imaging), but usually over a full circle. Data reconstruction takes into account the fact that the emitted rays are also attenuated within the patient, e.g., photons emanating from deep inside the patient are considerably attenuated by surrounding tissues. While in CT absorption is the essence of the imaging process, in SPECT attenuation degrades the images. Thus, data of the head reconstructed without attenuation correction may show substantial artificial enhancement of the peripheral brain structures relative to the deep ones. The simplest way to deal with this problem is to filter the data before reconstruction. A more elegant but elaborate method used in triple head cameras is to introduce a gamma-ray line source between two camera heads, which are detected by the opposing camera head after being partly absorbed by the patient. This camera head then yields transmission data while the other two collect emission data. Note that the camera collecting transmission data has to be fitted with a converging collimator to admit the appropriate gamma rays.

SPECT is routinely used in clinical studies. For example, SPECT is usually performed with a gamma camera comprising a collimator fixed on a gamma detector that traces a revolution orbit around the patient's body. The gamma rays, emitted by a radioactive tracer accumulated in certain tissues or organs of the patient's body, are sorted by the collimator and recorded by the gamma detector under various angles around the body. From the acquired planar images, the distribution of the activity inside the patient's body is computed using certain reconstruction algorithms. Generally, the so-called Expectation-Maximization of the Maximum-Likelihood (EM-ML) algorithm is used, as described by Shepp et al. (IEEE Trans. Med. Imaging 1982; 2:113-122) and by Lange et al. (J. Comput. Assist. Tomogr. 1984; 8:306-316). This iterative algorithm minimizes the effect of noise in SPECT images.

In preferred embodiments, the imaging agents of the present technology are used as imaging agents for PET imaging and SPECT imaging.

It is contemplated that the imaging agents of the present technology are provided to a nuclear pharmacist or a clinician in kit form.

A pharmaceutical composition produced according to the present technology comprises use of one of the aforementioned imaging agents and a carrier such as a physiological buffered saline solution a physiologically buffered sodium acetate carrier. It is contemplated that the composition will be systemically administered to the patient as by intravenous injection. Suitable dosages for use as a diagnostic imaging agent are, for example, from about 0.2 to about 2.0 mCi of 1-131 labeled imaging agent for the adrenal medulla or tumors therein, and from about 2.0 to about 10.0 mCi of the 1-123 labeled agent for imaging of the heart and adrenal medulla or tumors therein. For use as a therapeutic agent, a higher dosage is required, for example, from about 100 to about 300 mCi of the imaging agent material.

It will be appreciated by those skilled in the art that the imaging agents of the present technology are employed in accordance with conventional methodology in nuclear medicine in a manner analogous to that of the aforementioned imaging agents. Thus, a composition of the present technology is typically systemically applied to the patient, and subsequently the uptake of the composition in the selected organ is measured and an image formed, for example, by means of a conventional gamma camera.

Further understanding of use of the present technology can be obtained from the following examples and from Kline, et al.: “Myocardial Imaging in Man with [123 I]-Meta-Iodobenzylguanidine,” J. Nucl. Med. 22:129-132, 1981; Wieland, et al: “Myocardial Imaging with a Radioiodinated Norepinephrine Storage Analog,” J. Nucl. Med. 22:22-31, 1981; Valk, et al: “Spectrum of Pheochromocytoma in Multiple Endocrine Neoplasia: A Scintigraphic Portrayal Using .sup.131 I-Meta-Iodobenzylguanidine,” Ann. Intern. Med., Vol. 94, pp. 762-767 (1981); Sisson, et al.: “Scintigraphic Localization of Pheochromocytoma,” New Eng. J. Med., Vol. 305, pp. 12-17, (1981); and Lynn, et al., “Portrayal of Pheochromocytoma and Normal Human Adrenal Medulla by m-[I-123]-iodobenzylguanidine”, J. Nucl. Med., Vol. 25, Vol. 436-440 (1984); and U.S. Pat. Nos. 4,584,187 and 4,622,217; of these articles are specifically incorporated by reference herein.

In some embodiments, the imaging agents are used for early detection of AD in a subject.

Although the disclosure herein refers to certain illustrated embodiments, it is to be understood that these embodiments are presented by way of example and not by way of limitation.

### EXAMPLES

A variety of peptides are distributed throughout the central nervous system (CNS) where they have important neurological functions. The peptide Gonadotropin-Releasing Hormone (GnRH) and its receptors are found predominantly in the hippocampal region of the brain and have shown connection to early stages of Alzheimer's Disease (AD) development. Peptides are not efficiently transported across the blood-brain-barrier and, in addition, their limited metabolic stability make them unsuitable as radiolabeled imaging agents for use in the CNS. Thus, to circumvent the inherent low CNS penetration properties of GnRH peptides, small molecular GnRH antagonists were synthesized and labeled with fluorine-18.

### Example 1

### Biological Characteristics of Pilot Compounds

During the development of embodiments of the technology provided herein, two pilot compounds were synthesized based on a previously published library of furamide based compounds:

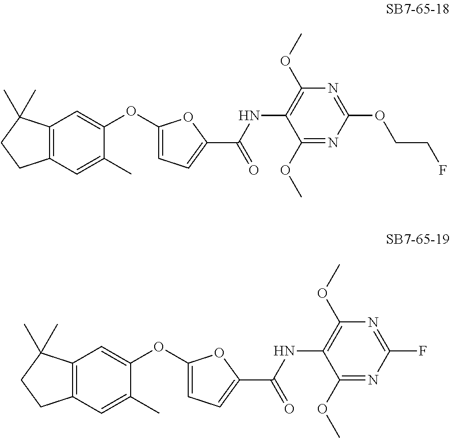 [SMILES: COc1nc(OCCF)nc(OC)c1NC(=O)c1ccc(Oc2cc3c(cc2C)CCC3(C)C)o1 ; COc1nc(F)nc(OC)c1NC(=O)c1ccc(Oc2cc3c(cc2C)CCC3(C)C)o1]

These compounds were then used as substrates for labeling with [¹⁸F]fluoride and to produce ¹⁹F-fluorinated reference standards (see, e.g., Li, et al (2006) J Med Chem 49: 3362-3367).

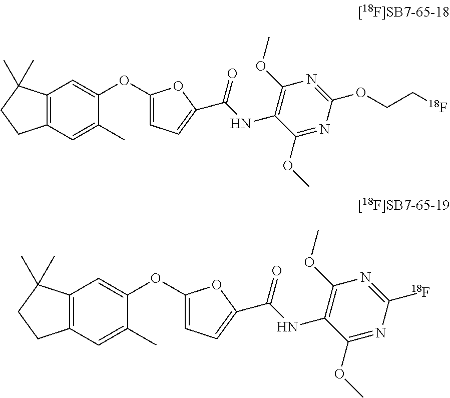 [SMILES: COc1nc(OCC[18F])nc(OC)c1NC(=O)c1ccc(Oc2cc3c(cc2C)CCC3(C)C)o1 ; COc1nc([18F])nc(OC)c1NC(=O)c1ccc(Oc2cc3c(cc2C)CCC3(C)C)o1]

The compounds were evaluated to determine their binding affinities, physicochemical properties, and radiochemical properties (see Table 1).

### Methods

PSA was calculated using ChemBioDraw Ultra 12.0; Clog D was experimentally determined; Kᵢ was determined by an in vitro assay in HEK 293 cells expressing rat GnRH receptor (see below); the radiochemical yield with respect to isotope decay was corrected based on the start of synthesis, and radiochemical purity was analyzed by Radio-HPLC.

For the membrane preparation and radioligand binding assays, HEK 293 cells were grown in Dulbecco's modified Eagle's medium with 10% fetal bovine serum, penicillin (100 U/ml), and streptomycin (100 μg/ml), and transiently transfected with rat GnRH receptor using LipofectAMINE 2000 (Invitrogen) according to the manufacturer's protocol. 48 hours later, membranes were prepared and radioligand binding was performed as previously described (see, e.g., Krobert et al (2001) “The cloned human 5-HT7 receptor splice variants: a comparative characterization of their pharmacology, function and distribution” Naunyn Schmiedebergs Arch Pharmacol 363(6): 620-32). Radioligand binding studies were performed with 0.18-0.32 nM [¹²⁵I] LH-RH Dtrp6 (K_{d}=0.3 nM) and increasing concentration of the indicated peptide. K_{d} of [¹²⁵I] LH-RH Dtrp6 was 0.3 nM, which was used to determine the Kᵢ of the peptides from the calculated IC₅₀.

### Results

The collected data are provided in Table 1;

TABLE 1
Binding affinity (Kᵢ), physiochemical, and radiochemical properties

| | | | | | Radio- | Radio- |
|---|---|---|---|---|---|---|
| | | PSA | CLog D | Kᵢ | chemical | chemical |
| Compound | Mw | (Å²) | (pH 7.4) | (nM) | yield (5) | purity (%) |
| [¹⁸F]SB7-65-18 | 485.2 | 100 | 1.22 | 1.36 | 6-10% | >95% |
| [¹⁸F]SB7-65-19 | 441.2 | 91 | 1.33 | 42 | 8-14% | >95% |

In Table 1, PSA was calculated using ChemBioDraw Ultra 12.0; Clog D was experimentally determined; Ki was determined by an in vitro assay in HEK 293 cells expressing rat GnRH receptor; the radiochemical yield with respect to isotope decay was corrected to the start of synthesis, and radiochemical purity was analyzed by Radio HPLC.

### Example 2

### Synthesis and Evaluation of [18F]Labeled GnRH Receptor Antagonists

During the development of embodiments of the technology provided herein, a competitive binding assay was used to measure the binding affinity to rat GnRH receptor of the two nonradioactive reference ¹⁹F-compounds described above. Radiolabeling was performed from [¹⁸F]fluoride using the corresponding mesylate and chloro-precursors using K[¹⁸F]/K222 complex in DMSO. Log P values and serum stability were investigated and their brain uptake was studied by small animal PET imaging in healthy rats.

Synthesized ¹⁹F-reference compounds displayed affinity (in the nM range) for the GnRH receptor when competed with [¹²⁵I][D-Trp⁶]-LHRH. Radiolabeling experiments yielded both compounds in 3-10% decay-corrected yield and the RPC was greater than 98% after final formulation. Log P values were 1.3 and 1.4 for [¹⁸F]SB7-65-18 and [¹⁸]SB7-65-19, respectively. [¹⁸F]SB7-65-18 was stable in rat serum over 2 hours but failed to show significant brain uptake. The more lipophilic [¹⁸F]SB7-65-19 entered the brain and was clearly visibly by PET (0.4% ID/g at 10 minutes); but, short serum stability (t_{1/2}<30 minutes) due to hydrolysis of the amide bond precludes its further use.

In sum, the data collected demonstrate the successful synthesis of two novel small molecular GnRH antagonists labeled with ¹⁸F and having nM affinity for the rat GnRH receptor. [¹⁸F]SB7-65-19 displayed significant brain uptake, thus showing the utility of this class of compounds as radiotracers for GnRH receptor imaging in CNS. Other related compounds provided herein and contemplated by the technology also find use as radiotracers for medical imaging.

### Example 3

### Quantification of Uptake in Brain

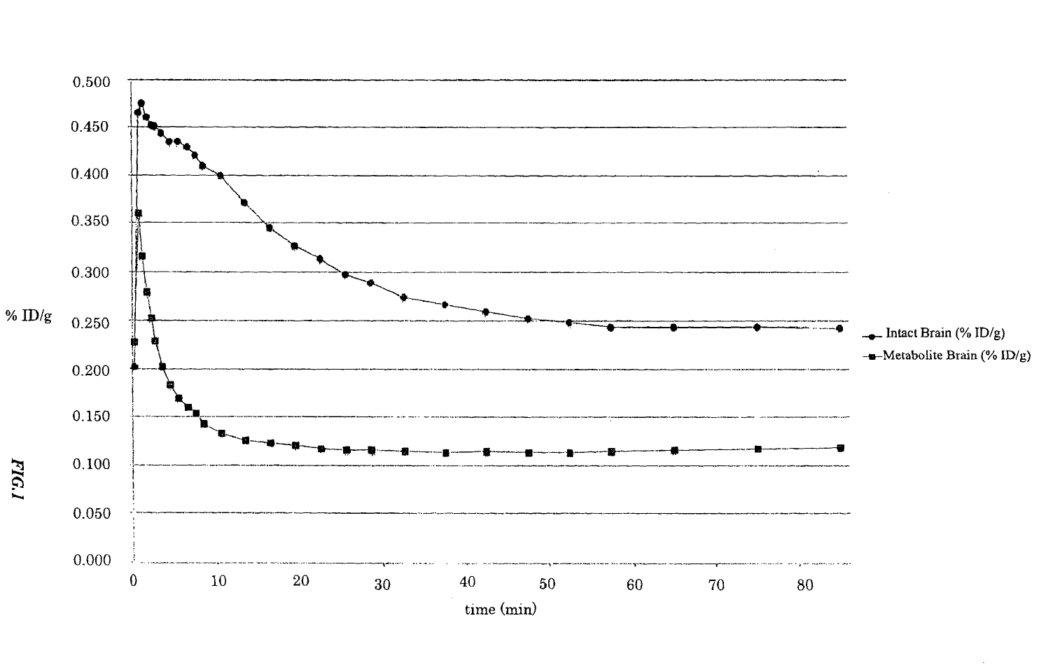

During the development of embodiments of the technology provided herein, experiments were conducted to measure the uptake of embodiments of the compounds described herein in brain tissue. In particular, time activity curves were obtained for uptake in intact brain and metabolite brain (FIG. 1).

### Example 4

### Chemical Syntheses

During the development of embodiments of the technology provided herein, several compounds were synthesized. In some embodiments, certain intermediate compounds were synthesized. Intermediates and end products were synthesized according to techniques known in the art and according to the following experimental procedures.

### Experimental

Solvents and chemicals were purchased from Aldrich (Milwaukee, Wis.), Fisher Scientific, or WVR unless stated otherwise. Sep-Pak SPE cartridges were obtained from Waters (Milford, Mass.) and ¹⁸F Trap & Release Columns were purchased from ORTG, Inc. (Oakdale, Tenn.). RP-HPLC was performed using Beckman-Coulter (Brea, Calif.) chromatography systems equipped with Jupiter Proteo C-12 columns (250×4.6 mm, 4 mm, Phenomenex, Torrance, Calif.) and single wave length or diode array UV detectors (e.g., for detection of signals at approximately 254 nm) connected in series to a Bioscan Flow Count photomultiplier tube (PMT) (Bioscan, Washington, D.C.). Mass spectrometry analysis was performed using a Thermo Electron LTQ-Orbitrap Hybrid MS spectrometer. NMR spectra were recorded using a Bruker Avance 600 or Bruker Avance 500 spectrometer. [¹⁸F]Fluoride was produced from the ¹⁸O(p,n)¹⁸F nuclear reaction on [¹⁸O]H₂O purchased from Marshall Isotopes Ltd. (Tel Aviv, Israel) using a CTI RDS 111 negative ion cyclotron (Knoxville, Tenn.). Total radioactivity was measured with a Capintec dose calibrator.

Reversed-phase HPLC was used to purify and analyze the products using a solvent A comprising 0.05% TFA in water (v/v) and a solvent B comprising acetonitrile.

HPLC systems were equipped with both a UV absorbance detector (e.g., 254 nm) and a radioactivity detector (PMT) connected in series, which accounts for the slight difference between detected retention times for corresponding ¹⁸F- and ¹⁹F-compounds.

Analytical HPLC system A: Phenomenex Jupiter 4μ Proteo 90 Å column (250×4.6 mm, 4 μm), solvent B isocratic 50% for 2 minutes, then linear gradient to 90% over 20 minutes, flow rate 1.5 mL/minute. Semi-preparative HPLC system B: Phenomenex Jupiter 10μ Proteo 90 Å (250×10 mm, 10 μm), solvent B isocratic 50% for 2 min, then linear gradient to 90% over 30 minutes, flow rate 3 mL/minute. Sep-Pak SPE cartridges were preconditioned according to the manufacturer's recommendations. The ¹⁸F-radiolabeled products were identified by co-injection with authentic reference ¹⁹F-compounds.

Radioligand binding studies were performed with 0.18-0.32 nM [¹²⁵I]LHRH-[D-Trp⁶] ([¹²⁵I]Triptorelin) titrated with increasing concentrations of compound and cell membranes expressing the human type I GnRH receptor (Bmax: 1.0 pmol/mg, Merck Millipore) The K_{d} of [¹²⁵I]LHRH-[D-Trp⁶] was 0.24 nM, which was used to determine the Kᵢ values from the calculated IC₅₀.

Octanol/water partition coefficients were determined as follows. Approximately 10 kBq of ¹⁸F-compounds in 50 μL PBS were diluted in 450 μL of PBS (pH 7.4) and added to 500 μL of n-octanol in an Eppendorf tube (n=3). After vortexing for 3 minutes, the tubes were centrifuged (10,300 rpm for 6 minutes) and 100 μL aliquots of the PBS and n-octanol phases were carefully transferred to separate tubes. The radioactivity of the samples was counted in a γ-counter.

Serum and in vivo stability measurements were performed as follows. Approximately 5 MBq of ¹⁸F compounds (e.g., peptides or small molecules) in PBS (50 μL) was added to freshly collected rat serum (0.4 mL) and samples were incubated at 37° C. in Eppendorf tubes. After 1 hour and 2 hours, ice-cold ethanol (400 μL) was added and the mixtures were centrifuged at 13,400 rpm for 10 minutes. The resulting supernatants were diluted with Solvent A and analyzed by radio-HPLC.

### Chemistry

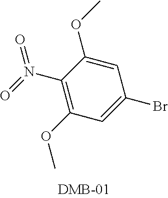

### 5-bromo-1,3-dimethoxy-2-nitrobenzene

DMB-01 was prepared according to International Patent Application WO 2011/094186 A1 (Vernier J-M, “Derivatives of 4-(N-azacycloalkyl) anilides as potassium channel modulators”). ¹H NMR (600 MHz, DMSO): δ 7.17 (s, 2H), δ 3.89 (s, 6H); ¹³C NMR (151 MHz, DMSO) δ 151.51, 130.33, 124.87, 109.22, 108.88, 57.32; HRESIMS: No ionization of compound with ESI.

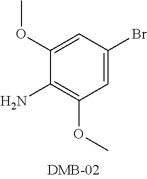

### 4-bromo-2,6-dimethoxyaniline

Confirms correct mass of DMB-01 indirectly. Fe(s) mediated reduction of DMB-01 in aqueous-ethanolic ammonium chloride solution. Compound gives correct mass for aniline by ESI-MS and expected isotopic distribution pattern of bromine containing compound. HRESIMS [M+H]⁺ m/z 231.9971 (calculated for C₈H₁₀BrNO₂, 231.9968).

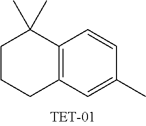

### 1,1,6-trimethyl-1,2,3,4-tetrahydronaphthalene

Tetralin TET-01 was prepared according to Parlow J J, “Selective syntheses of substituted 6-alkyl-1,1-dimethyl-1,2,3,4-tetrahydronaphthalenes” Tetrahedon, 49: 2577-2588 (1993). ¹H NMR (600 MHz, CDCl₃): δ 7.25 (d, 1H, J=8 Hz), δ 6.98 (d, 1H, J=8 Hz), δ 6.90 (bs, 1H), δ 2.76 (t, 2H), δ 2.31 (t, 3H), δ 1.83 (m, 2H), δ 1.69 (m, 2H), δ 1.31 (s, 6H); ¹³C NMR (151 MHz, CDCl₃): δ 143.02, δ 136.13, δ 134.71, δ 129.74, δ 126.86, δ 126.69, δ 39.65, δ 33.68, δ 32.05, δ 32.05, δ 30.87, δ 20.96, δ 19.98. HRESIMS: No ionization of compound with ESI.

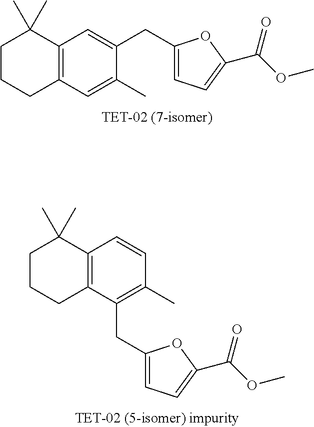 [SMILES: COC(=O)c1ccc(Cc2cc3c(cc2C)CCCC3(C)C)o1 ; COC(=O)c1ccc(Cc2c(C)ccc3c2CCCC3(C)C)o1]

### Methyl 5-((3,8,8-trimethyl-5,6,7,8-tetra-hydronaphthalen-2-yl)methyl)furan-2-carboxylate

TET-02 was isolated after alkaline hydrolysis of the methyl ester. The Friedel-Craft alkylation of TET-01 with methyl 5-(chloromethyl)-2-furoate with anhydrous aluminum trichloride as catalyst was investigated in various solvents (dichloromethane, nitro methane, 1,2-dichloroethane) and temperatures to produce preferentially a 7-position alkylation of the tetrahydronaphthalene backbone in TET-01. However, all investigated conditions yielded a 1:1 mixture of the 7- and 5 alkylation products.

To a solution containing TET-01 (0.5 g, 2.87 mmol) and methyl 5-(chloromethyl)-2-furoate (0.418 g, 2.39 mmol) in nitro methane (10 ml) was added aluminum trichloride (0.383 g, 2.87 mmol) slowly under N₂ gas. The solution was stirred at room temperature for 48 hours. The reaction was quenched with ice-cold water and the crude product extracted with ethyl acetate (2×100 ml). The organic phases were combined and washed with brine, dried over MgSO₄, and concentrated under vacuum. The crude product was purified by silica gel chromatography using hexanes/ethyl acetate (19/1 v/v) to afford 0.302 g (40%) of TET-02 (mixture of 5- and 7-regioisomers) as viscous oil. HRESIMS: No ionization of compound with ESI.

Isomers co-eluted both on normal phase silica gel and reversed phased (C18) HPLC. Mixture was hydrolyzed with 4 M NaOH in THF to yield the acids and an improved reversed phase HPLC separation of the two regioisomers.

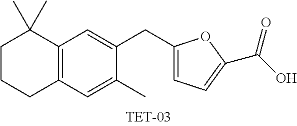 [SMILES: Cc1cc2c(cc1Cc1ccc(C(=O)O)o1)C(C)(C)CCC2]

### 5-((3,8,8-trimethyl-5,6,7,8-tetrahydronaphthalen-2-yl)methyl)furan-2-carboxylic acid

TET-02 regioisomers were dissolved in THF (10 mL) and 4 M NaOH (10 mL). The reaction mixture was stirred overnight and quenched with 1 M HCl to an acidic pH. The reaction was then extracted with dichloromethane and concentrated. The mixture of isomers was dissolved in a 1:1 mixture of mobile phase A and B and subjected to revered-phased preparative HPLC using an isocratic method (61% solvent B in A at a flow rate of 7 ml/minute using a preparative reversed-phased C18 column (Phenomenex Jupiter C18 10 μm, 250×21.2 mm) to afford 100 mg of isomerically pure TET-03. ¹H NMR (600 MHz, CDCl₃): δ 7.22 (d, J=3.5, 1H), δ 7.13 (s, 1H), δ 6.87 (s, 1H), δ 5.97 (d, J=3.5, 1H), δ 4.00 (s, 2H), δ 2.27 (t, 2H), δ 2.20 (s, 3H), δ 1.78 (m, 2H), δ 1.64 (m, 2H) δ 1.25 (s, 6H); ¹³C NMR (151 MHz, CDCl₃): δ 163.35, δ 161.52, δ 143.97, δ 142.37, δ 135.28, δ 133.41, δ 132.10, δ 131.15, δ 128.27, δ 121.70, δ 109.28, δ 39.42, δ 33.65, δ 32.75, δ 32.00 (s), δ 32.00 (s) δ 30.40, δ 19.86, δ 18.98; HMBC and HSQC confirm the correct regioisomer; HRESIMS [M+H]⁺ m/z 299.1645 (calculated for C₁₉H₂₂O₃, 299.1642).

In some embodiments, compounds IND-01, IND-022 IND-03, IND-04, and IND-05 were synthesized (see, e.g., as described above, e.g., for the following structures).

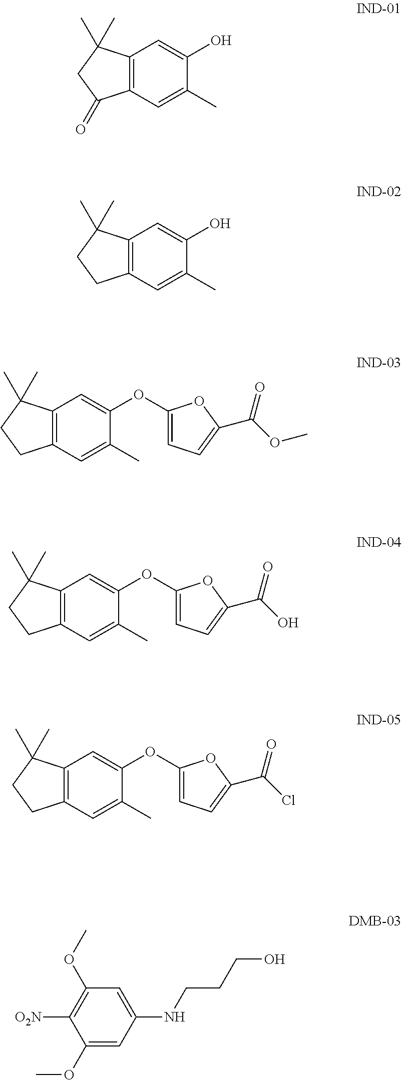

### 3-((3,5-dimethoxy-4-nitrophenyl)amino)propan-1-ol

Cs₂CO₃ (684 mg, 2.1 mmol), (±BINAP) 2,2′-bis(diphenylphosphino)-1,1′-binaphthalene (56 mg, 0.1 mmol) and palladium(II) acetate (6.7 mg, 0.03 mmol) in 1 mL 1,4-dioxane were stirred under argon in a 25 mL Schlenk tube at 70° C. for 10 minutes, after which the reaction acquired a deep red color. DMB-01 (300 mg, 1.2 mmol) and 3-amino-1-propanol (135 mg, 1.8 mmol) in 3 mL 1,4-dioxane were added to the catalytic complex under a counter stream of argon. The sealed reaction mixture was stirred for 16 hours at 110° C., cooled, and diluted with 10 ml ethyl acetate. After filtration through celite and concentration, the residue was purified by flash chromatography (ethyl acetate, neat) affording DMB-03 as a yellow oil (180 mg, 58%). ¹H NMR (600 MHz, CDCl₃): δ 5.80 (s, 2H), δ 3.82 (t, J=5.7, 2H), δ 3.80 (s, 6H), δ 3.30 (t, J=6.5, 2H), δ 1.89 (p, J=6.3, 2H); ¹³C NMR (151 MHz, CDCl₃): δ 154.40, δ 150.98, δ 123.73, δ 88.74, δ 61.20, δ 56.37, δ 42.00, δ 31.30; HRESIMS [M+H]⁺ m/z 257.1128 (calculated for C₁₁H₁₆N₂O₅, 257.1132).

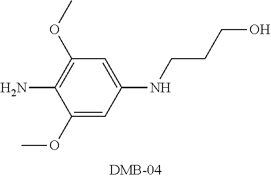 [SMILES: COc1cc(NCCCO)cc(OC)c1N]

### 3-((4-amino-3,5-dimethoxyphenyl)amino)propan-1-ol

DMB-03 (100 mg, 0.4 mmol), Fe(s) (125 mg), and NH₄Cl (125 mg) were stirred vigorously in a mixture of H₂O/MeOH (12 mL, 2:10) at RT for 2 hours. The spent iron was filtered off and the filtrate was concentrated. The residue was dissolved in dichloromethane and the ammonium chloride was filtered off. After concentration, the residue was purified by flash chromatography (ethyl acetate, neat) affording DMB-04 as a black viscous oil (25 mg, 28%). The compound was highly unstable and was used immediately in the following reaction. NMR on this product was not feasible. HRESIMS [M+H]⁺ m/z 227.1389 (calculated for C₁₁H₁₈N₂O₃, 227.1390).

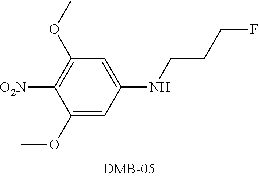 [SMILES: COc1cc(NCCCF)cc(OC)c1[N+](=O)[O-]]

### N-(3-fluoropropyl)-3,5-dimethoxy-4-nitroaniline

Cs₂CO₃ (500 mg, 1.5 mmol), (±BINAP) 2,2′-bis(diphenylphosphino)-1,1′-binaphthalene (38 mg, 0.06 mmol) and palladium(II) acetate (5 mg, 0.02 mmol) in 1 mL 1,4-dioxane were stirred under argon in a 25 mL Schlenk tube at 70° C. for 10 minutes, which acquired a deep red color. DMB-01 (200 mg, 0.8 mmol) and 3-fluoro-propylamine hydrochloride (132 mg, 1.2 mmol) in 2 mL 1,4-dioxane were added to the catalytic complex under a counter stream of argon. The sealed reaction mixture was stirred for 5 hours at 80° C., cooled, and diluted with 10 ml ethyl acetate. After filtration through celite and concentration, the residue was purified by flash chromatography (40% ethyl acetate in hexanes) affording DMB-05 as an oil (180 mg, 44%). ¹H NMR (600 MHz, CDCl₃): δ 5.76 (s, 2H), δ 4.62 (t, J=5.5, 1H), δ 4.55 (t, J=5.5, 1H), δ 3.80 (s, 6H), δ 3.33 (t, J=6.7, 2H), δ 2.03 (m, 1H), δ 1.99 (m, 1H); ¹³C NMR (151 MHz, CDCl₃): δ 154.36, 154.11, 151.17, 123.70, 100.09, 97.73, 97.72, 88.46, 88.27, 88.04, 82.78, 81.69, 64.47, 56.31, 40.31, 40.28, 36.72, 30.73, 30.24, 30.11; HRESIMS [M+H]⁺ m/z 259.1087 (calculated for C₁₁H₁₅FN₂O₄, 259.1089).

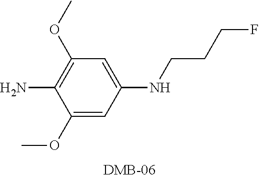 [SMILES: COc1cc(NCCCF)cc(OC)c1N]

### N¹-(3-fluoropropyl)-3,5-dimethoxybenzene-1,4-diamine

DMB-05 (92 mg, 0.36 mmol), Few (200 mg), EtOH (3 mL), and saturated NH₄Cl (3 mL) were stirred vigorously at RT for 3 hours. The spent iron was filtered off and the filtrate was concentrated, neutralized with a saturated sodium bicarbonate solution, and extracted with dichloromethane. After concentration, the residue was purified by flash chromatography (ethyl acetate, neat) affording DMB-06 as an oil (64 mg, 78%). ¹H NMR (600 MHz, CDCl₃): δ 5.87 (s, 2H), δ 4.63 (t, J=5.5, 1H), δ 4.55 (t, J=5.5, 1H), δ 3.78 (s, 6H), δ 3.75 (t, 2H), δ 1.99 (m, 2H); ¹³C NMR (151 MHz, CDCl₃): δ 148.14, 140.28, 119.73, 98.47, 83.12, 82.03, 55.95, 55.84; HRESIMS [M+H]⁺ m/z 229.1350 (calculated for C₄₄H₄₇FN₂O₂, 229.1347).

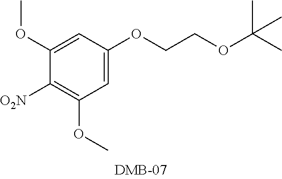 [SMILES: COc1cc(OCCOC(C)(C)C)cc(OC)c1[N+](=O)[O-]]

### 5-(2-(tert-butoxy)ethoxy)-1,3-dimethoxy-2-nitrobenzene

To a stirred solution of Cs₂CO₃ (391 mg, 1.2 mmol), 5-di(1-adamantylphosphino)-1-(1,3,5-triphenyl-1H-pyrazol-4-yl)-1H pyrazole (11 mg, 0.017 mmol), and palladium(II) acetate (1.8 mg, 1 mol %) in 2 mL dry toluene under argon in a 25 mL Schlenk tube at 80° C., DMB-01 (200 mg, 0.8 mmol) and 2-tert-butoxyethanol (284 mg, 2.4 mmol) were added. The sealed reaction mixture was stirred at 80° C. overnight. After cooling, the reaction mixture was diluted with 10 ml ethyl acetate and filtered through celite and concentrated. The residue was purified by flash chromatography (20% ethyl acetate in hexanes) affording DMB-07 as an oil (156 mg, 68%). ¹H NMR (600 MHz, CDCl₃): δ 6.17 (s, 2H), δ 4.11 (t, J=5.3, 2H), δ 3.84 (s, 6H), δ 3.72 (t, J=5.3, 2H), δ 1.24 (s, 9H); ¹³C NMR (151 MHz, CDCl₃) δ 161.78, 153.39, 91.68, 73.71, 70.06, 68.80, 60.43, 56.54, 27.63; HRESIMS [M+H]⁺ m/z 300.1442 (calculated for C₄₄H₂₁NO₆, 300.1442).

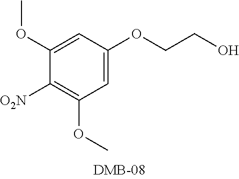 [SMILES: COc1cc(OCCO)cc(OC)c1[N+](=O)[O-]]

### 2-(3,5-dimethoxy-4-nitrophenoxy)ethanol

DMB-07 (60 mg, 0.2 mmol) was stirred for 48 hours in a solution of 85% phosphoric acid (3 mL) and toluene (1 mL). The reaction mixture was neutralized and extracted with dichloromethane (3×10 mL). The combined organic phases were dried over Na₂SO₄ and concentrated to afford DMB-08 as an oil (39 mg, 80%). ¹H NMR (600 MHz, CDCl₃) δ 6.15 (s, 2H), 4.11 (t, J=4.5, 2H), 3.99 (t, J=4.5, 2H), 3.85 (s, 6H); ¹³C NMR (151 MHz, CDCl₃) δ 161.33, 153.43, 106.13, 91.44, 69.86, 61.31, 56.56; HRESIMS [M+H]⁺ m/z 244.0812 (calculated for C₁₀H₁₃NO₆, 244.0816).

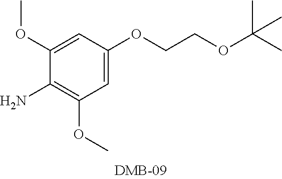 [SMILES: COc1cc(OCCOC(C)(C)C)cc(OC)c1N]

### 4-(2-(tert-butoxy)ethoxy)-2,6-dimethoxyaniline

DMB-07 (156 mg, 0.52 mmol), Few (200 mg) in EtOH (4 mL) and saturated NH₄Cl (3 mL) was stirred vigorously at room temperature for 3 hours. The spent iron was filtered off, the filtrate was neutralized with 1 M NaOH, and then diluted with 30 mL H₂O. The aqueous phase was extracted with dichloromethane (3×10 mL) and ethyl acetate (3×10 mL). The combined organic phases were dried over Na₂SO₄ and concentrated affording DMB-09 as an oil (139 mg, 99%). ¹H NMR (600 MHz, CDCl₃): δ 6.18 (s, 2H), 4.06 (t, J=5.4, 2H), 3.86 (s, 6H), 3.71 (t, J=5.4, 2H), 1.24 (s, 9H); ¹³C NMR (151 MHz, CDCl₃) δ 157.64, 154.29, 105.34, 91.98, 73.64, 68.62, 60.53, 56.34, 27.63; HRESIMS [M+H]⁺ m/z 270.1702 (calculated for C14H23NO4, 270.1700).

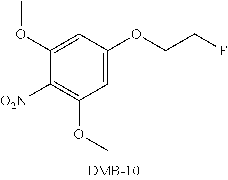 [SMILES: COc1cc(OCCF)cc(OC)c1[N+](=O)[O-]]

### 5-(2-fluoroethoxy)-1,3-dimethoxy-2-nitrobenzene

To a stirred solution of DMB-008 (44.5 mg, 0.18 mmol) in dry dichloromethane (4 mL) cooled to 0° C. was added DAST (diethylaminosulfur trifluoride) (30 mg, 3 eq.) dropwise over 1 minute. The reaction mixture was stirred for 1 hour at 0° C., allowed to react at room temperature, and then quenched with saturated sodium bicarbonate solution. The reaction mixture was extracted with dichloromethane (3×10 mL), the combined organic phases were washed with water and dried over MgSO₄. Following concentration, the residue was purified by flash chromatography (60% ethyl acetate in hexanes) affording DMB-10 as an oil (30 mg, 65%). ¹H NMR (600 MHz, CDCl₃): δ 6.16 (s, 2H), 4.81 (m, 1H), 4.73 (m, 1H), 4.27 (m, 1H), 4.22 (m, 1H), 3.86 (s, 6H); ¹³C NMR (151 MHz, CDCl₃): δ 161.04, 153.47, 91.62, 82.22, 81.08, 67.85, 67.71, 56.61; HRESIMS [M+H]⁺ m/z 246.0769 (calculated for C₁₀H₁₂FNO₅, 246.0773).

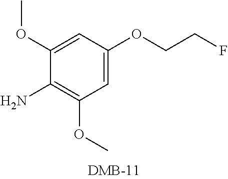

### 4-(2-fluoroethoxy)-2,6-dimethoxyaniline

DMB-10 (30 mg, 0.12 mmol), Few (120 mg) in EtOH (3 mL), and saturated NH₄Cl (3 mL) were stirred vigorously at room temperature for 3 hours. The spent iron was filtered off, the filtrate was neutralized with sodium bicarbonate, and then diluted with 30 mL H₂O. The aqueous phase was extracted with ethyl acetate (3×10 mL). The combined organic phases were dried over Na₂SO₄ and concentrated affording DMB-11 as an oil (18 mg, 68%). ¹H NMR (600 MHz, CDCl₃): δ 6.16 (s, 2H), 4.80 (m, 1H), 4.72 (m, 1H), 4.26 (m, 1H), 4.21 (m, 1H), 3.85 (s, 6H); ¹³C NMR (151 MHz, CDCl₃): δ 151.36, 148.09, 119.65, 92.73, 82.80, 81.67, 68.44, 68.31, 56.02; HRESIMS [M+H]⁺ m/z 216.1031 (calculated for C₁₀H₁₄FNO₃, 216.1031).

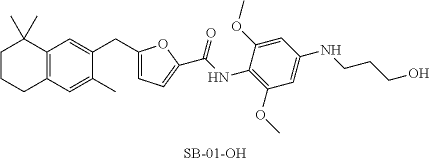 [SMILES: COc1cc(NCCCO)cc(OC)c1NC(=O)c1ccc(Cc2cc3c(cc2C)CCCC3(C)C)o1]

### N-(4-((3-hydroxypropyl)amino)-2,6-dimethoxyphenyl)-5-((3,8,8-trimethyl-5,6,7,8-tetrahydronaphthalen-2-yl)methyl)furan-2-carboxamide

A stirred vial was charged with acid TET-03 (26 mg, 0.1 mmol, 1 eq.), HATU (1-[bis(dimethylamino)methylene]H-1,2,3-triazolo[4,5-b]pyridinium 3-oxid hexafluorophosphate) (34 mg, 1 eq), and DIPEA (N,N-diisopropylethylamine (37 μL, 3 eq.) in DMF (1 mL). After 5 minutes, amine DMB-04 (24 mg, 0.09 mmol) was added. After 1 h reaction time HPLC indicated a complete reaction (full consumption of acid). The reaction mixture was diluted with water and extracted with ethyl acetate (3×5 mL). The combined organic phases were washed with water and dried over Na₂SO₄. Following removal of organic solvent in vacuo, the residue was purified by flash chromatography (ethyl acetate, neat) affording SB-01-OH as a yellowish solid (25 mg, 54%). ¹H NMR (600 MHz, CDCl₃): δ 8.02 (s, 1H), 7.10 (s, 2H), 7.09 (d, J=3.5, 1H), 6.88 (s, 1H), 6.55 (s, 1H), 6.01 (d, J=3.5, 1H), 3.84 (t, 2H), 3.80 (s, 6H), 3.48 (s, 2H), 3.41 (t, 2H), 2.71 (t, 2H), 2.08 (s, 3H), 2.01 (m, 2H), 1.79 (m, 2H), 1.64 (m, 2H), 1.24 (s, 6H); ¹³C NMR (151 MHz, CDCl₃) δ 175.33, 162.84, 157.76, 157.36, 156.41, 155.90, 146.55, 143.93, 135.18, 133.31, 132.54, 131.11, 128.00, 116.55, 109.28, 96.11, 60.30, 56.41, 50.96, 39.43, 38.76, 36.70, 33.64, 32.51, 31.99, 31.64, 30.38, 29.45, 20.75, 19.87, 19.00; HRESIMS [M+H]⁺ m/z 507.2854 (calculated for C₃₀H₃₈N₂O₅, 507.2854).

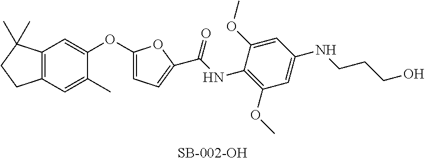 [SMILES: COc1cc(NCCCO)cc(OC)c1NC(=O)c1ccc(Oc2cc3c(cc2C)CCC3(C)C)o1]

### N-(4-((3-hydroxypropyl)amino)-2,6-dimethoxyphenyl)-5-((3,3,6-trimethyl-2,3-dihydro-1 H-inden-5-yl)oxy)furan-2-carboxamide

A stirred vial was charged with acid IND-04 (12 mg, 0.04 mmol, 1 eq.), HATU (1-[bis(dimethylamino)methylene]-1H-1,2,3-triazolo[4,5-b]pyridinium 3-oxid hexafluorophosphate) (15.9 mg, 1 eq), and DIPEA N diisopropylethylamine (22 μL, 3 eq.) in DMF (1 mL); after 5 minutes, amine DMB-04 (11 mg, 0.05) was added. After a 1-hour reaction time, HPLC indicated a complete reaction (full consumption of acid). The reaction mixture was diluted with water and extracted with ethyl acetate (3×5 mL). The combined organic phases were washed with water and dried over Na₂SO₄. Following removal of organic solvent in vacuo, the residue was purified by flash chromatography (ethyl acetate, neat) affording SB-02-OH as a yellowish solid (16 mg, 77%). ¹H NMR (600 MHz, CDCl₃); δ 7.10 (s, 2H), 7.09 (d, 1H), 6.88 (s, 1H), 6.55 (s, 1H), 5.29 (d, 1H), 3.89 (t, 2H), 3.80 (s, 6H), 3.41 (t, 2H), 2.71 (t, 2H), 2.84 (t, 2H), 2.22 (s, 3H), 1.94 (t, 2H), 1.76 (m, 2H), 1.23 (s, 6H); HRESIMS [M+H]⁺ m/z 495.2479 (calculated for C₂₈H₃₄N₂O₆, 495.2490).

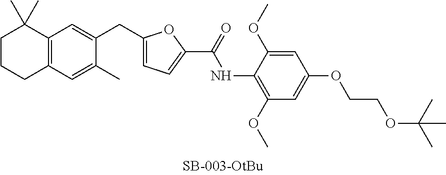 [SMILES: COc1cc(OCCOC(C)(C)C)cc(OC)c1NC(=O)c1ccc(Cc2cc3c(cc2C)CCCC3(C)C)o1]

### N-(4-(2-(tert-butoxy)ethoxy)-2,6-dimethoxyphenyl)-5-((3,8,8-trimethyl-5,6,7,8-tetrahydronaphthalen-2-yl)methyl)furan-2-carboxamide

A stirred vial was charged with acid TET-03 (24 mg, 0.08 mmol, 1 eq.), HATU (1-[Bis(dimethylamino)methylene]-1H-1,2,3-triazolo[4,5-b]pyridinium 3-oxid hexafluorophosphate) (32 mg, 1 eq), and DIPEA (N,N-diisopropylethylamine (44 μL, 3 eq.) in DMF (1 mL). After 5 minutes, amine DMB-09 (23 mg, 0.09 mmol) was added. After a 1-hour reaction time, HPLC indicated a complete reaction (full consumption of acid). The reaction mixture was diluted with water and extracted with ethyl acetate (3×5 mL). The combined organic phases were washed with water and dried over Na₂SO₄. Following removal of organic solvent in vacuo, the residue was purified by flash chromatography (1:1 ethyl acetate/hexanes) affording SB-03-OtBU as a glassy solid (22 mg, 48%). ¹H NMR (600 MHz, CDCl₃) δ 7.26 (d, 1H), 7.10 (s, 1H), 6.87 (s, 1H), 6.22 (s, 2H), 5.99 (d, 1H), 4.09 (t, 2H), 3.96 (s, 2H), 3.79 (s, 6H), 3.72 (t, 2H), 2.71 (t, 2H), 2.24 (s, 3H), 1.79 (m, 2H), 1.64 (m, 2H), 1.24 (s, 6H), 1.24 (s, 3H); ¹³C NMR (151 MHz, CDCl₃) δ 159.53, 156.46, 143.87, 135.08, 133.35, 132.72, 131.07, 128.01, 109.19, 106.57, 92.00, 73.58, 68.51, 60.56, 56.19, 39.45, 33.64, 32.52, 32.00, 30.38, 27.66, 19.88, 19.04; HRESIMS [M+H]⁺ m/z 550.3145 (calculated for C₃₃H₄₃NO₆, 550.3163).

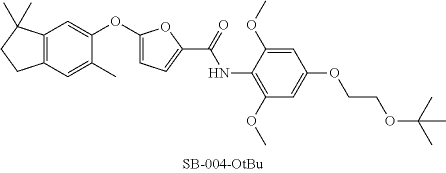 [SMILES: COc1cc(OCCOC(C)(C)C)cc(OC)c1NC(=O)c1ccc(Oc2cc3c(cc2C)CCC3(C)C)o1]

### N-(4-(2-(tert-butoxy)ethoxy)-2,6-dimethoxyphenyl)-5-((3,3,6-trimethyl-2,3-dihydro-1H-inden-5-yl)oxy)furan-2-carboxamide

To a stirred vial charged with acid chloride IND-05 (34 mg, 0.11 mmol, 1.2 eq.) and DIPEA (N,N-diisopropylethylamine (50 μL, 3 eq.) in dry dichloromethane (1 mL) at 0° C., amine DMB-09 (25 mg, 0.09 mmol) in dichloromethane (0.5 mL) was added dropwise. After a 5-hour reaction time, HPLC indicated a complete reaction (full consumption of amine). The reaction mixture was diluted with ethyl acetate (20 mL) and washed with 0.1 M HCl, brine, and water. The organic phase was dried over Na₂SO₄. Following removal of organic solvent in vacuo, the residue was purified by flash chromatography (7:3 ethyl acetate/hexanes) affording SB-04-OtBU as a glassy solid (30 mg, 62%). ¹H NMR (600 MHz, CDCl₃) δ 7.20 (bs, 1H), 7.12 (d, J=3.5, 1H), 7.06 (s, 1H), 6.85 (s, 1H), 6.22 (s, 2H), 5.29 (d, J=3.5, 1H), 4.09 (t, J=5.6, 2H), 3.80 (s, 6H), 3.72 (t, J=5.6, 2H), 2.85 (t, J=7.2, 2H), 2.25 (s, 3H), 1.94 (t, J=7.2, 2H), 1.25 (s, 9H), 1.22 (s, 6H); ¹³C NMR (151 MHz, CDCl₃) δ 159.52, 156.47, 152.38, 152.10, 140.01, 127.37, 126.82, 112.70, 106.64, 92.04, 87.94, 73.56, 68.49, 60.55, 56.14, 44.25, 41.88, 29.69, 28.67, 27.65, 16.01; HRESIMS [M+H]⁺ m/z 538.2803 (calculated for C₃₁H₃₉NO₇, 538.2800).

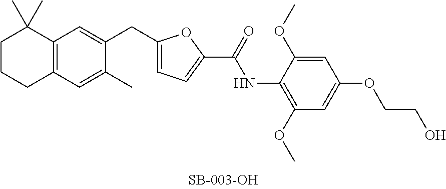 [SMILES: COc1cc(OCCO)cc(OC)c1NC(=O)c1ccc(Cc2cc3c(cc2C)CCCC3(C)C)o1]

### N-(4-(2-hydroxyethoxy)-2,6-dimethoxyphenyl)-5-((3,8,8-trimethyl-5,6,7,8-tetrahydronaphthalen-2-yl)methyl)furan-2-carboxamide

SB-003-OtBu (22 mg, 0.04 mmol) was stirred vigorously at 50° C. for 24 hours in a solution of 85% phosphoric acid (2 mL) and toluene (1 mL). The reaction mixture was quenched with 0.1 M NaOH, diluted with water (10 mL), and extracted with dichloromethane (3×5 mL). The organic phases were combined and washed with water (5 mL) and dried over Na₂SO₄. After removal of organic solvent in vacuo, SB-003-OH was used further without purification. ¹H NMR (600 MHz, CDCl₃) δ 7.47 (bs, 1H), 7.11 (d, 1H), 6.89 (s, 1H), 6.21 (s, 1H), 6.05 (d, 1H), 5.30 (s, 2H), 4.08 (t, 2H), 4.00 (t, 2H), 3.99 (s, 2H) 3.83 (s, 6H), 2.72 (t, 2H), 2.25 (s, 3H), 1.79 (m, 2H), 1.65 (m, 2H), 1.26 (s, 6H); ¹³C NMR (151 MHz, CDCl₃) δ 158.31, 143.94, 135.22, 133.28, 131.12, 127.99, 91.90, 61.85, 39.42, 33.62, 32.00, 30.35, 19.84, 19.05; HRESIMS [M+H]⁺ m/z 494.2512 (calculated for C₂₉H₃₅NO₆, 494.2537).

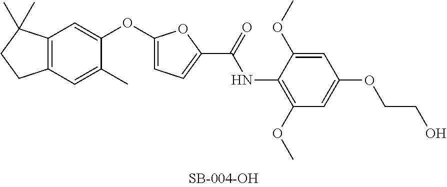 [SMILES: COc1cc(OCCO)cc(OC)c1NC(=O)c1ccc(Oc2cc3c(cc2C)CCC3(C)C)o1]

### N-(4-(2-hydroxyethoxy)-2,6-dimethoxyphenyl)-5-((3,3,6-trimethyl-2,3-dihydro-1H-inden-5-yl)oxy)furan-2-carboxamide

SB-004-OtBu (30 mg, 0.06 mmol) was stirred vigorously at 50° C. for 24 hours in a solution of 85% phosphoric acid (2 mL) and toluene (1 mL). The reaction mixture was quenched with 0.1 M NaOH, diluted with water (10 mL), and extracted with dichloromethane (3×5 mL). The organic phases were combined and washed with water (5 mL) and dried over Na₂SO₄. After removal of organic solvent in vacuo, the SB-004-OH was used further without purification. ¹H NMR (600 MHz, CDCl₃) δ 7.17 (d, 1H), 7.01 (s, 1H), 6.95 (s, 1H), 6.17 (s, 2H), 5.30 (d, 1H), 4.08 (t, 2H), 3.98 (t, 2H), 3.79 (s, 6H), 3.72 (t, 2H), 2.20 (s, 3H), 1.88 (t, 2H(, 1.21 (s, 6H); HRESIMS [M+H]⁺ m/z 482.2166 (calculated for C₂₇H₃₁NO₇, 482.2174).

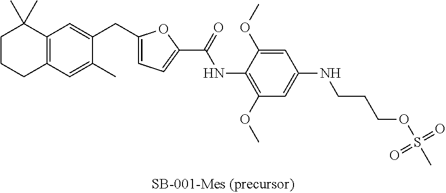 [SMILES: COc1cc(NCCCOS(C)(=O)=O)cc(OC)c1NC(=O)c1ccc(Cc2cc3c(cc2C)CCCC3(C)C)o1]

### 3-((3,5-dimethoxy-4-(5-((3,8,8-trimethyl-5,6,7,8-tetrahydronaphthalen-2-yl)methyl)furan-2-carboxamido)phenyl)amino)propyl methanesulfonate

To a stirred vial under an argon atmosphere cooled to 0° C. containing SB-001-0H (25 mg, 0.05 mmol) and triethylamine (21 μL, 0.15 mmol, 3 eq.) in dichloromethane (3 mL), methanesulfonyl chloride (5 μL, 0.06 mmol, 1.2 eq) was added. After a 1-hour reaction time, HPLC and TLC indicated a complete reaction. Following removal of organic solvent in vacuo, the residue was purified by silica flash chromatography (ethyl acetate, neat) affording SB-001-Mes as a greasy solid (12 mg, 41%). ¹H NMR (600 MHz, CD₃CN): δ 7.49 (s, 1H), 7.23 (s, 1H), 6.98 (d, J=3.5, 1H), 6.73 (s, 1H), 6.12 (d, J=3.5, 1H), 5.95 (s, 2H), 4.33 (t, 2H), 3.99 (s, 2H), 3.76 (s, 6H), 3.27 (t, 2H), 3.04 (s, 3H), 2.69 (t, 2H), 2.17 (s, 3H), 1.98 (m, 2H), 1.77 (m, 2H), 1.65 (m, 2H), 1.24 (s, 6H); ¹³C NMR (151 MHz, CD₃CN): δ 158.85, 158.37, 158.20, 157.31, 150.33, 144.42, 140.19, 135.75, 134.15, 134.10, 131.69, 128.97, 115.63, 109.56, 109.36, 106.47, 89.83, 69.59, 68.78, 57.00, 56.28, 40.32, 37.42, 34.19, 32.55, 32.05, 30.81, 29.46, 29.06, 20.48, 18.99; HRESIMS [M+H]⁺ m/z 585.2623 (calculated for C₃₁H₄0N₂O₇S, 585.2629).

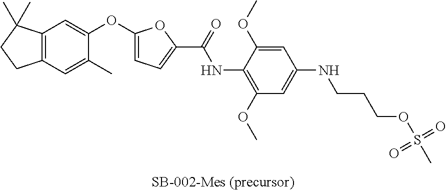 [SMILES: COc1cc(NCCCOS(C)(=O)=O)cc(OC)c1NC(=O)c1ccc(Oc2cc3c(cc2C)CCC3(C)C)o1]

### 3-((3,5-dimethoxy-4-(5-((3,3,6-trimethyl-2,3-dihydro-1H-inden-5-yl)oxy)furan-2-carboxamido)phenyl)amino)propyl methanesulfonate

To a stirred vial under an argon atmosphere cooled to 0° C. containing SB-002-OH (18 mg, 0.04 mmol) and triethylamine (15 μL, 0.11 mmol, 3 eq.) in dichloromethane (3 mL), methanesulfonyl chloride (10 μL, 0.13 mmol, 3 eq) was added. After a 1-hour reaction time, HPLC and TLC indicated a complete reaction. Following removal of organic solvent in vacuo, the residue was purified by silica flash chromatography (ethyl acetate, neat) affording SB-002-Mes as a greasy solid (10 mg, 49%). ¹H NMR (600 MHz, MeOD): δ 7.11 (d, J=3.5, 1H), 7.10 (s, 1H), 6.89 (s, 1H), 5.98 (s, 2H), 5.30 (d, J=3.5, 1H), 4.37 (t, J=6.1, 2H), 3.77 (s, 6H), 3.29 (t, 2H), 3.07 (s, 3H), 2.86 (t, J=7.2, 2H), 2.23 (s, 3H), 2.05 (m, 2H), 1.95 (t, J=7.2, 2H), 1.23 (s, 6H); ¹³C NMR (151 MHz, MeOD) δ 161.76, 158.51, 153.53, 141.38, 128.33, 127.99, 118.29, 113.86, 90.25, 88.26, 69.62, 56.15, 45.14, 42.86, 40.67, 37.03, 30.36, 30.07, 28.84, 15.88; HRESIMS [M+H]⁺ m/z 573.2254 (calculated for C₂₉H₃₆N₂O₈S, 573.2265).

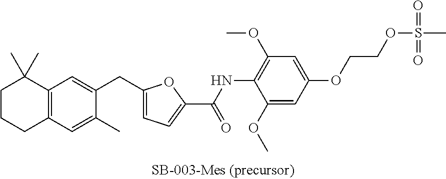 [SMILES: COc1cc(OCCOS(C)(=O)=O)cc(OC)c1NC(=O)c1ccc(Cc2cc3c(cc2C)CCCC3(C)C)o1]

### 2-(3,5-dimethoxy-4-(5-((3,8,8-trimethyl-5,6,7,8-tetrahydronaphthalen-2-yl)methyl)furan-2-carboxamido)phenoxy)ethyl methanesulfonate

To a stirred vial under an argon atmosphere cooled to 0° C. containing SB-003-OH (20 mg, 0.04 mmol) and triethylamine (15 μL, 0.11 mmol, 3 eq.) in dichloromethane (3 mL), methanesulfonyl chloride (12 μL, 0.15 mmol, 3 eq) was added. After a 1-hour reaction time, HPLC and TLC indicated a complete reaction. Following removal of organic solvent in vacuo, the residue was purified by silica flash chromatography (70% ethyl acetate in hexanes) affording SB-003-Mes as a glassy solid (12 mg, 51%). ¹H NMR (600 MHz, CDCl₃): δ 7.28 (s, 1H), 7.10 (s, 1H), 7.08 (d, J=3.1, 1H), 6.88 (s, 1H), 6.19 (s, 2H), 6.01 (d, J=3.1, 1H), 4.58 (m, 2H), 4.27 (m, 2H), 3.97 (s, 2H), 3.81 (s, 6H), 3.10 (s, 3H), 2.72 (t, J=6.3, 2H), 2.24 (s, 3H), 1.78 (m, 2H), 1.64 (m, 2H), 1.25 (s, 6H); ¹³C NMR (151 MHz, CDCl₃) δ 158.47, 157.21, 156.63, 146.96, 143.88, 135.11, 133.34, 132.70, 131.07, 127.97, 116.10, 109.21, 107.50, 91.93, 67.86, 66.39, 56.22, 39.44, 38.04, 33.65, 32.49, 32.00, 30.38, 19.88, 19.03. HRESIMS [M+H]⁺ m/z 572.2309 (calculated for C₃₀H₃₇NO₈S, 572.2313).

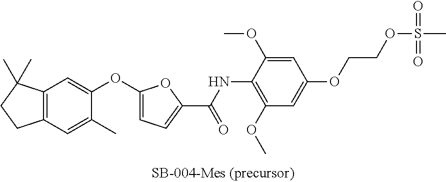 [SMILES: COc1cc(OCCOS(C)(=O)=O)cc(OC)c1NC(=O)c1ccc(Oc2cc3c(cc2C)CCC3(C)C)o1]

### 2-(3,5-dimethoxy-4-(5-((3,3,6-trimethyl-2,3-dihydro-1H-inden-5-yl)oxy)furan-2-carboxamido)phenoxy)ethyl methanesulfonate

To a stirred vial under an argon atmosphere cooled to 0° C. containing SB-004-OH (20 mg, 0.04 mmol) and triethylamine (18 μL, 0.11 mmol, 3 eq.) in dichloromethane (2 mL), methanesulfonyl chloride (8 μL, 0.23 mmol, 5 eq) was added. After a 1-hour reaction time, HPLC and TLC indicated a complete reaction. Following removal of organic solvent in vacuo, the residue was purified by silica flash chromatography (80% ethyl acetate in hexanes) affording SB-004-Mes as a glassy solid (20 mg, 85%). ¹H NMR (600 MHz, CDCl₃); δ 7.19 (s, 1H), 7.12 (d, J=3.5, 1H), 7.07 (s, 1H), 6.85 (s, 1H), 6.19 (s, 2H), 5.30 (d, J=3.5, 1H), 4.58 (m, 2H), 4.27 (m, 2H), 3.82 (s, 6H), 3.10 (s, 3H), 2.85 (t, J=7.2, 2H), 2.26 (s, 3H), 1.94 (t, J=7.2, 2H), 1.23 (s, 6H); ¹³C NMR (151 MHz, CDCl₃) δ 159.60, 158.46, 156.66, 152.42, 152.09, 140.07, 139.12, 127.40, 126.83, 117.61, 112.71, 107.61, 92.02, 87.93, 67.86, 66.43, 56.25, 44.27, 41.89, 38.06, 29.70, 28.68, 16.02; HRESIMS [M+H]⁺ m/z 560.1952 (calculated for C₂₈H₃₃NO₉S, 560.1949).

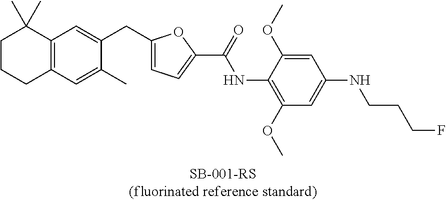 [SMILES: COc1cc(NCCCF)cc(OC)c1NC(=O)c1ccc(Cc2cc3c(cc2C)CCCC3(C)C)o1]

### N-(4-((3-fluoropropyl)amino)-2,6-dimethoxyphenyl)-5-((3,8,8-trimethyl-5,6,7,8-tetrahydronaphthalen-2-yl)methyl)furan-2-carboxamide

A stirred vial was charged with acid TET-03 (4 mg, 0.01 mmol, 1 eq.), HATU (1-[bis(dimethylamino)methylene]-1H-1,2,3-triazolo[4,5-b]pyridinium 3-oxid hexafluorophosphate) (5 mg, 0.01 mmol, 1 eq), and DIPEA (N,N-diisopropylethylamine (7 μL, 0.04 mmol, 3 eq.) in DMF (1 mL). After 1 minute, amine DMB-06 (4 mg, 0.02 mmol) was added. After a 1-hour reaction time, HPLC indicated a complete reaction (full consumption of acid). The reaction mixture was diluted with water containing 0.05% TFA and purified by reversed-phase preparative HPLC (isocratic 70% solvent A at 3.5 ml/min). The fraction containing pure SB-001-RS was isolated by lyophilization as a white solid, (2.1 mg, 4.1 μmol, 41%). ¹H NMR (600 MHz, CD₃CN) δ 7.49 (s, 1H), 7.23 (s, 1H), 6.95 (d, J=3.1, 1H), 6.87 (s, 1H), 6.12 (d, J=3.1, 1H), 5.94 (s, 2H), 4.62 (t, J=5.8, 2H), 4.54 (t, J=5.8, 2H), 3.99 (s, 2H), 3.71 (s, 6H), 3.26 (t, J=6.9, 2H), 2.70 (t, J=6.4, 2H), 2.24 (s, 3H), 2.01 (m, 2H), 1.77 (m, 2H), 1.65 (m, 2H), 1.25 (s, 6H). ¹³C NMR (151 MHz, CD₃CN) δ 158.32, 158.13, 150.47, 147.99, 144.37, 135.70, 134.10, 134.04, 131.61, 128.91, 115.57, 109.30, 89.70, 83.70, 82.63, 46.86, 39.97, 34.13, 32.49, 31.98, 30.75, 20.42, 18.93; HRESIMS [M+H]⁺ m/z 509.2804 (calculated for C₃₀H₃₇FN₂O₄, 509.2810).

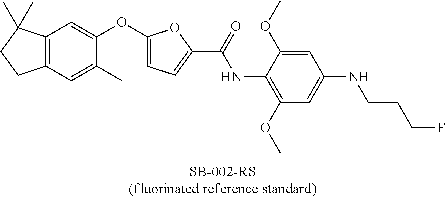 [SMILES: COc1cc(NCCCF)cc(OC)c1NC(=O)c1ccc(Oc2cc3c(cc2C)CCC3(C)C)o1]

### N-(4-((3-fluoropropyl)amino)-2,6-dimethoxyphenyl)-5-((3,3,6-trimethyl-2,3-dihydro-1H-inden-5-yl)oxy)furan-2-carboxamide

A vial was charged with acid IND-04 (11 mg, 0.04 mmol, 1 eq.), HATU (1-[bis(dimethylamino)methylene]-1H-1,2,3-triazolo[4,5-b]pyridinium 3-oxid hexafluorophosphate) (15 mg, 0.04 mmol, 1 eq), and DIPEA (N,N-diisopropylethylamine) (20 μL, 0.12 mmol, 3 eq.) in DMF (300 μL). After 3 minutes, amine DMB-06 (8 mg, 0.04 mmol) was added. After a 1-hour reaction time, HPLC indicated a complete reaction (full consumption of acid). The reaction mixture was diluted with water containing 0.05% TFA and purified by reversed-phase preparative HPLC (isocratic 70% solvent A at 3.5 ml/minutes). The fraction containing pure SB-002-RS was isolated by lyophilization as a white solid, (5 mg, 10 μmol, 25%). ¹H NMR (600 MHz, CD₃CN): δ 7.42 (bs, 1H), 7.12 (s, 1H), 6.99 (bs, 1H), 6.95 (s, 1H), 5.97 (s, 2H), 5.36 (d, J=3.5, 1H), 4.61 (t, J=5.8, 2H), 4.54 (t, J=5.8, 2H), 3.72 (s, 6H), 3.27 (t, J=6.9, 2H), 2.86 (t, J=7.2, 2H), 2.24 (s, 3H), 1.99 (m, 2H), 1.21 (s, 6H); ¹³C NMR (151 MHz, CD₃CN): δ 160.46, 158.22, 153.32, 153.04, 141.08, 128.15, 127.65, 113.76, 90.37, 88.66, 83.72, 82.65, 56.33, 44.82, 42.40, 30.79, 30.66, 30.08, 28.64, 15.90; HRESIMS [M+H]⁺ m/z 497.2442 (calculated for C₂₈H₃₃FN₂O₅, 497.2447).

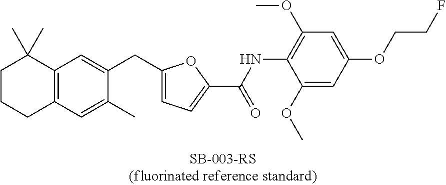 [SMILES: COc1cc(OCCF)cc(OC)c1NC(=O)c1ccc(Cc2cc3c(cc2C)CCCC3(C)C)o1]

### N-(4-(2-fluoroethoxy)-2,6-dimethoxyphenyl)-5-((3,8,8-trimethyl-5,6,7,8-tetrahydronaphthalen-2-yl)methyl)furan-2-carboxamide

A stirred vial was charged with acid TET-03 (5 mg, 0.02 mmol, 1 eq.), HATU (1-[bis(dimethylamino)methylene]-1H-1,2,3-triazolo[4,5-b]pyridinium 3-oxid hexafluorophosphate) (6.1 mg, 0.02 mmol, 1 eq), and DIPEA (N,N-diisopropylethylamine (14 μL, 0.08 mmol, 4 eq.) in DMF (1 mL). After 1 minute, amine DMB-11 (3.5 mg, 0.02 mmol) was added. After a 1-hour reaction time, HPLC indicated a near complete reaction (full consumption of acid). The reaction mixture was diluted with water containing 0.05% TFA and purified by reversed-phase preparative HPLC (isocratic 70% solvent A at 3.5 ml/min). The fraction containing pure SB-003-RS was isolated by lyophilization as a white solid, (5.4 mg, 10.1 μmol, 50%). ¹H NMR (600 MHz, CDCl₃): δ 7.39 (bs, 1H), 7.12 (s, 1H), 7.10 (bs, 1H), 6.88 (s, 1H), 6.20 (s, 2H), 6.02 (d, J=3.1, 1H), 4.80 (m, 1H), 4.72 (m, 1H), 4.25 (m, 1H), 4.20 (m, 1H), 3.97 (s, 2H), 3.80 (s, 6H), 2.72 (t, J=6.3, 2H), 2.24 (s, 3H), 1.79 (m, 2H), 1.64 (m, 2H), 1.25 (s, 6H); ¹³C NMR (151 MHz, CDCl₃): δ 159.25, 156.58, 143.93, 135.19, 133.33, 132.53, 131.12, 127.99, 116.96, 109.43, 91.82, 82.52, 81.39, 67.66, 67.53, 56.13, 39.43, 33.65, 32.51, 32.00, 30.39, 19.88, 19.03; HRESIMS [M+H]⁺ m/z 496.2490 (calculated for C₂₉H₃₄FNO₅, 496.2494).

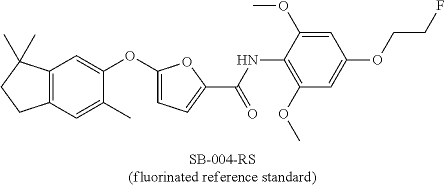 [SMILES: COc1cc(OCCF)cc(OC)c1NC(=O)c1ccc(Oc2cc3c(cc2C)CCC3(C)C)o1]

### N-(4-(2-fluoroethoxy)-2,6-dimethoxyphenyl)-5-((3,3,6-trimethyl-2,3-dihydro-1H-inden-5-yl)oxy)furan-2-carboxamide

A stirred vial was charged with acid chloride IND-05 (6.1 mg, 0.02 mmol) and triethylamine (10 μL, 0.08) in dry dichloromethane (1.5 mL) at 0° C. Then, amine DMB-11 (4 mg, 0.02 mmol) in dichloromethane (0.5 mL) was added dropwise. After 1 hour, the organic solvent was removed in vacuo, the residue was taken up in a mixture of water containing 0.05% TFA/acetonitrile (1:1), and purified by reversed-phase preparative HPLC (isocratic 70% solvent A at 3.5 ml/min). The fraction containing pure SB-004-RS was isolated by lyophilization as a white solid, (3.2 mg, 7 μmol, 33%). ¹H NMR (600 MHz, CDCl₃); δ 7.33 (bs, 1H), 7.18 (s, 1H), 7.07 (bs, 1H), 6.86 (s, 1H), 6.20 (s, 2H), 5.30 (d, J=3.6, 1H), 4.80 (m, 1H), 4.73 (m, 1H), 4.25 (m, 1H), 4.20 (m, 1H), 3.82 (s, 6H), 2.86 (t, J=7.2, 2H), 2.25 (s, 3H), 1.94 (t, J=7.2, 2H), 1.23 (s, 6H); ¹³C NMR (151 MHz, CDCl₃) δ 159.29, 156.58, 152.49, 151.83, 140.30, 127.45, 126.89, 118.96, 112.87, 91.77, 88.00, 82.52, 81.38, 67.64, 56.12, 44.27, 41.85, 29.71, 28.67, 16.02; HRESIMS [M+H]⁺ m/z 484.2129 (calculated for C₂₇H₃₀FNO₆, 484.2130).

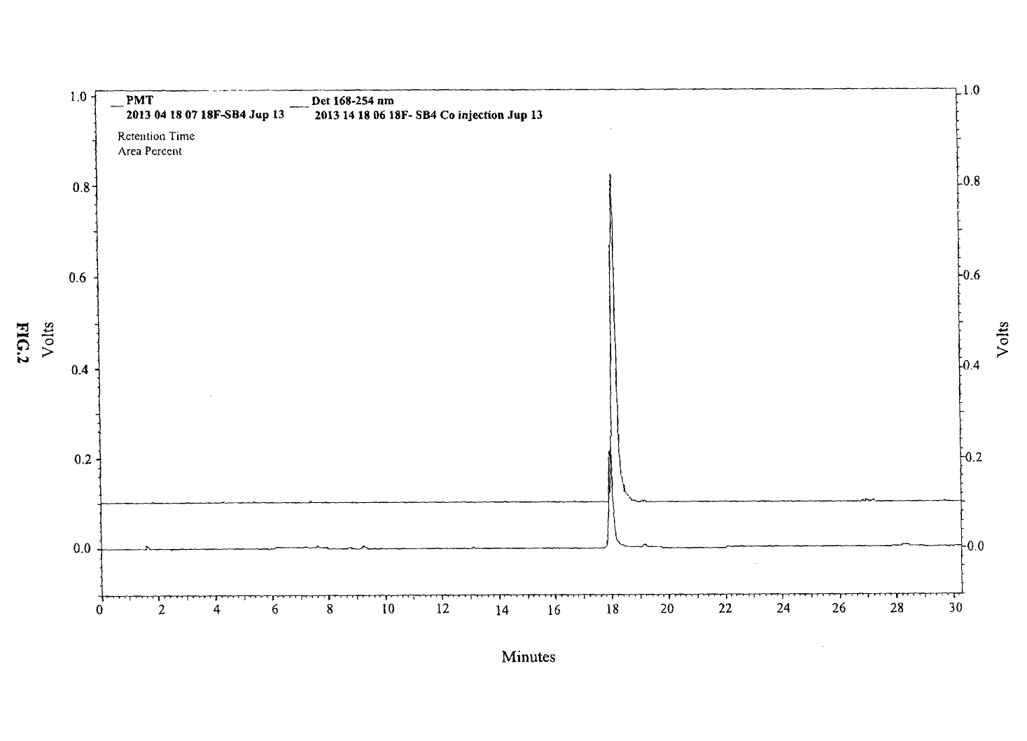

### Radiochemistry

[¹⁸F]fluoride (from 15 to 2000 mCi) was captured on a ¹⁸F-fluoride Trap & Release column cartridge and eluted with a solution of 4,7,13,16,21,24-hexaoxa-1,10-diazabicyclo[8.8.8]hexacosane (K222; 15 mg)/potassium carbonate (3 mg) in acetonitrile/water (1.5 mL; 94/6 v/v). The acetonitrile was evaporated and the K¹⁸F/K222 complex was dried by azeotropic distillation of the water using additional volumes (1 mL×3) of acetonitrile. A solution of mesylate precursor (1 mg) in anhydrous DMSO (0.3 mL) was added to the dried complex and the resulting mixture was heated to 100° C. (10 minutes). The reaction mixture was diluted with 1 mL solvent A and the radiolabeled product was purified by semi-preparative HPLC. The fraction containing ¹⁸F-labeled product (3 mL) was diluted with water and trapped on a C18-Sep-Paklight (Waters Inc.). The Sep-Pak was rinsed with water (10 mL) and dried with 10 mL air. Radioactivity was eluted with 1 mL absolute ethanol and the ethanol was reduced to a volume of 100 μL by heating to 50° C. under a gentle stream of N₂ gas. The residue was diluted with 1 to 2 mL of 0.9% sterile sodium chloride solution, which was used for the log P studies described herein. The identities of the radiolabelled products were confirmed by spiking the radioactive solution with the authentic ¹⁹F-reference standards and confirming co-elution by Radio-HPLC (see, e.g., FIG. 2).

Radiochemical yields for were 5.5%, 16%, 8.4%, and 12%, respectively (synthesis time of 2 hours, yields not corrected for decay, and n=3).

During the development of embodiments of the technology provided herein, the synthesized compounds were characterized and data were collected.

Log P values for compounds [¹⁸F]SB-001-RS, [¹⁸F]SB-002-RS, [¹⁸F]SB-003-RS, and [¹⁸F]SB-004-RS were 1.43, 1.49, 1.68, and 1.82, respectively (n=3). Log P values were measured in a phosphate-buffered saline (pH 7.4)/n-octanol system.

The receptor affinities (10 of compounds SB-001-RS, SB-002-RS, SB-003-RS, and SB-004-RS for the human GnRH receptor were measured (n=3) to be 3.7±2.3, 1.5±3.9, 0.6±0.4, and 2.1±2.1, respectively (Kᵢ provided in nM±SD).

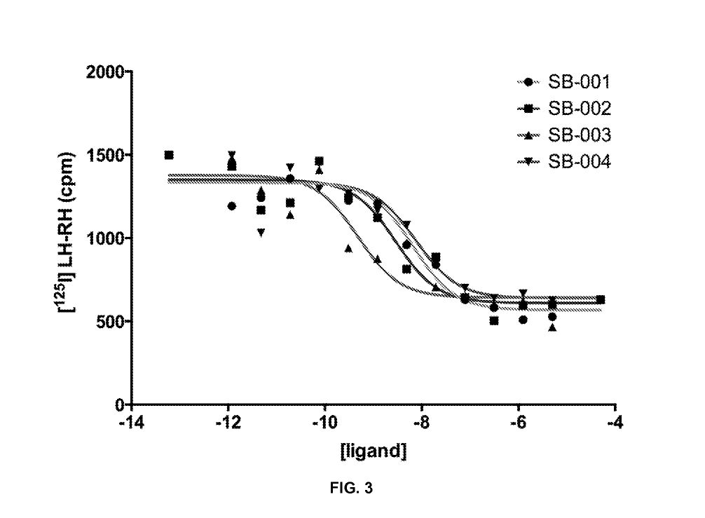

Further, binding curves for of compounds SB-001-RS, SB-002-RS, SB-003-RS, and SB-004-RS for the human GnRH receptor in competition for [¹²⁵I]Triptorelin were measured (n=3) (FIG. 3).

Also, the stabilities of the compounds were measured in rat serum and in vivo in rat blood and urine (Table 2). Table 2 provides the fraction (as a percentage of the initial amount) of compound remaining in serum, blood, or urine as a function of time (e.g., after 1 and 2 hours).

TABLE 2
% Intact 18F-compound

| | Serum | Serum | Blood | Urine | Urine |
|---|---|---|---|---|---|
| Compound | 1 h | 2 h | 1 h | 1 h | 2 h |
| [¹⁸F]SB-001-RS | >95 | 88.5 | 81 | 94.5 | NA |
| [¹⁸F]SB-002-RS | 99.1 | 97.5 | 97 | 99 | NA |
| [¹⁸F]SB-003-RS | >95 | 91.1 | 88.1 | 18.5 | NA |
| [¹⁸F]SB-004-RS | >95 | 97.3 | 97.5 | 99 | NA |

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A compound having the structure according to one of the following: 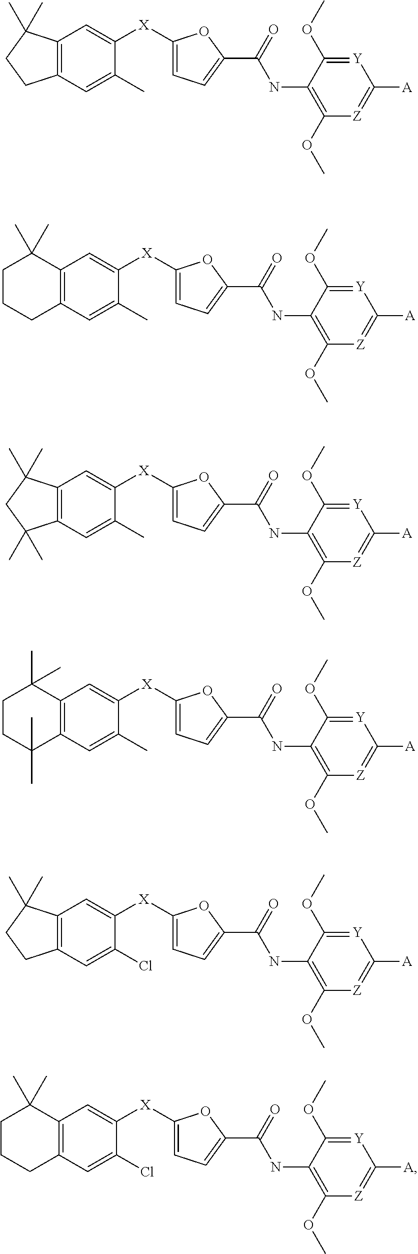
or a salt, a free base, or a combination thereof, wherein X is O or C; Y is C or N; Z is C or N; and A is one of the following:

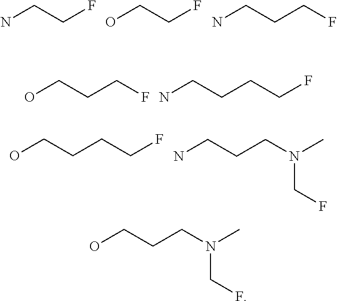

2. The compound according to claim 1 wherein the compound has a structure according to one of the following:

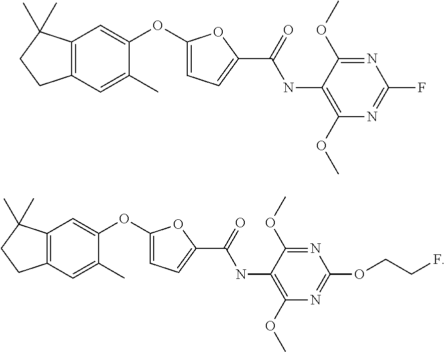 [SMILES: COc1nc(F)nc(OC)c1NC(=O)c1ccc(Oc2cc3c(cc2C)CCC3(C)C)o1 ; COc1nc(OCCF)nc(OC)c1NC(=O)c1ccc(Oc2cc3c(cc2C)CCC3(C)C)o1]

3. The compound according to claim 1 wherein F is ¹⁹F or ¹⁸F.

4. A method of imaging a subject, the method comprising administering a compound according to claim 1 and imaging the patient using positron emission tomography.

5. The method of claim 4 wherein the subject has or is suspected of having Alzheimer's disease.

6. The method of claim 4 wherein the subject has or is suspected of having a condition associated with the activity of gonadotropin-releasing hormone or a gonadotropin-releasing hormone receptor.

7. A method of imaging a tissue comprising contacting a tissue to be imaged with a compound according to claim 1 and imaging the tissue.

8. The method of claim 7 wherein the tissue is nervous tissue.

9. The method of claim 7 wherein the tissue is central nervous system tissue.

10. The method of claim 7 wherein the tissue is brain tissue.

11. The method of claim 7 wherein the tissue comprises a gonadotropin-releasing hormone receptor.

12. The method of claim 7 wherein the tissue comprises, or is suspected of comprising, a disease-associated plaque.

13. A composition comprising a compound according to claim 1 and a pharmaceutically acceptable carrier suitable for administration to a subject.

14-15. (canceled)

16. A compound having a structure according to one of the following: 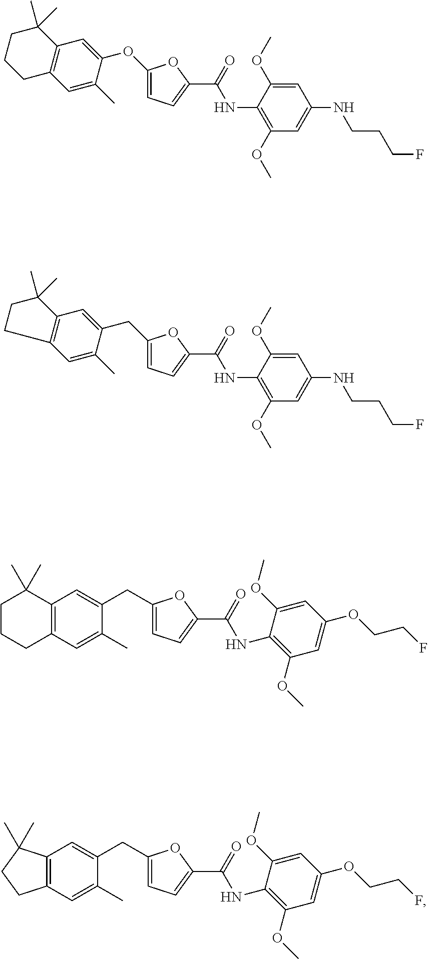 [SMILES: COc1cc(NCCCF)cc(OC)c1NC(=O)c1ccc(Oc2cc3c(cc2C)CCCC3(C)C)o1 ; COc1cc(NCCCF)cc(OC)c1NC(=O)c1ccc(Cc2cc3c(cc2C)CCC3(C)C)o1 ; COc1cc(OCCF)cc(OC)c1NC(=O)c1ccc(Cc2cc3c(cc2C)CCCC3(C)C)o1 ; COc1cc(OCCF)cc(OC)c1NC(=O)c1ccc(Cc2cc3c(cc2C)CCC3(C)C)o1]
or a salt, a free base, or a combination thereof.

17. The compound according to claim 16 wherein F is ¹⁹F or ¹⁸F.

18. The compound according to claim 16 having a Log P value in a phosphate-buffered saline (pH 7.4) and n-octanol system that is from 1.2 to 2.0.

19. The compound according to claim 16 having a receptor affinity (Kᵢ) for the human GnRH receptor of 0.1 to 6.0 nM.

20-21. (canceled)

22. The compound of claim 1 wherein A is H, alkyl, aryl, alkylaryl, amino, or alkoxy.
